(19) United States
(12) Patent Application Publication
(10) Pub. No.: US 2026/0317267 A1
(43) Pub. Date: Oct. 15, 2026

(54) SYSTEMS AND METHODS FOR GENERATION OF HYPERPOLARIZED MATERIALS

(71) Applicant: NVISION IMAGING TECHNOLOGIES GMBH, Ulm (DE)

(72) Inventors: Ilai SCHWARTZ, Neu-Ulm (DE); Stephan KNECHT, Stuttgart (DE)

(73) Assignee: NVISION IMAGING TECHNOLOGIES GMBH, Ulm (DE)

(21) Appl. No.: 19/111,069

(22) PCT Filed: Sep. 12, 2023
(86) PCT No.: PCT/IB2023/059051

§ 371 Date: Mar. 12, 2025

Related U.S. Application Data
(60) Provisional application No. 63/375,392, filed on Sep. 13, 2022.

(51) Int. Cl. C07C 69/738; A61K 49/10; C07B 59/00; C07C 69/732

(52) U.S. Cl. CPC C07C 69/738; A61K 49/10; C07B 59/001; C07C 69/732; C07B 2200/05

(57) ABSTRACT

The present disclosure describes hyperpolarized materials for use in nuclear magnetic resonance, magnetic resonance imaging, or similar applications. The present disclosure describes methods for producing hyperpolarized materials for use in nuclear magnetic resonance, magnetic resonance imaging, or similar applications. The present disclosure describes precursor compounds for use in producing hyperpolarized materials for use in nuclear magnetic resonance, magnetic resonance imaging, or similar applications.

### CROSS-REFERENCE

The present application claims priority to U.S. Provisional Application No. 63/375,392, entitled “SYSTEMS AND METHODS FOR GENERATION OF HYPERPOLARIZED MATERIALS,” filed on Sep. 13, 2022, which is incorporated herein by reference in its entirety for all purposes.

### TECHNICAL FIELD

The disclosed embodiments generally relate to the generation and purification of hyperpolarized materials for use in nuclear magnetic resonance, magnetic resonance imaging, or similar applications.

### BACKGROUND

Parahydrogen induced polarization (PHIP) is a method for polarizing metabolites for hyperpolarized (HP) Magnetic Resonance Imaging (MRI), with low cost and high throughput. Parahydrogen induced polarization with sidearm hydrogenation (PHIP-SAH) can be used to polarize metabolites, e.g., acetate molecules. However, existing PHIP-SAH polarization approaches may be unsuitable for preclinical or clinical HP MRI applications.

### BRIEF DESCRIPTION OF THE DRAWINGS

The accompanying drawings, which comprise a part of this specification, illustrate several embodiments and, together with the description, serve to explain certain principles and features of the disclosed embodiments. In the drawings:

FIG. 1 depicts a first exemplary process for generating polarized biorelevant imaging agents, in accordance with various embodiments.

FIG. 2 depicts a second exemplary process for generating polarized biorelevant imaging agents, in accordance with various embodiments.

FIG. 3 depicts a third exemplary process for generating polarized biorelevant imaging agents, in accordance with various embodiments.

FIG. 4 depicts a fourth exemplary process for generating polarized biorelevant imaging agents, in accordance with various embodiments.

FIG. 5 shows exemplary singlet lifetimes for (Z)-tert-butyl 4-((2-oxopropanoyl)oxy)but-2-enoate, (Z)-tert-butyl 4-((2-oxopropanoyl)oxy)but-2-enoate-D1, and (Z)-tert-butyl 4-((2-oxopropanoyl)oxy)but-2-enoate-D2, in accordance with various embodiments.

FIG. 6A shows an exemplary polarization transfer pulse sequence used for transferring spin order from the parahydrogen-associated protons in (Z)-tert-butyl 4-((2-oxopropanoyl)oxy)but-2-enoate, (Z)-tert-butyl 4-((2-oxopropanoyl)oxy)but-2-enoate-D1, and (Z)-tert-butyl 4-((2-oxopropanoyl)oxy)but-2-enoate-D2 to the natural abundance ¹³C nuclei present in (Z)-tert-butyl 4-((2-oxopropanoyl)oxy)but-2-enoate, (Z)-tert-butyl 4-((2-oxopropanoyl)oxy)but-2-enoate-D1, and (Z)-tert-butyl 4-((2-oxopropanoyl)oxy)but-2-enoate-D2, in accordance with various embodiments.

FIG. 6B shows exemplary ¹³C polarizations achieved for (Z)-tert-butyl 4-((2-oxopropanoyl)oxy)but-2-enoate-D2 using a variety of first time periods t_{sweep} in the polarization transfer pulse sequence of FIG. 6A, in accordance with various embodiments.

FIG. 7A shows exemplary ¹³C polarization levels for (Z)-tert-butyl 4-((2-oxopropanoyl)oxy)but-2-enoate-D2 following the polarization transfer procedure of FIGS. 6A and 6B, in accordance with various embodiments.

FIG. 8 shows exemplary ¹³C polarization levels for a deuterated parahydrogenated ester sidearm derivative of lactate, in accordance with various embodiments.

FIG. 9 shows exemplary ¹³C polarization levels for a deuterated parahydrogenated derivative of mono ethyl ketoglutarate, in accordance with various embodiments.

FIG. 10 shows exemplary ¹³C polarization levels for a deuterated parahydrogenated derivative of Z-OMPD mono methyl ester, in accordance with various embodiments.

### DETAILED DESCRIPTION

Reference will now be made in detail to exemplary embodiments, discussed with regards to the accompanying drawings. Unless otherwise defined, technical and/or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Recent work in the field of nuclear magnetic resonance (NMR) and magnetic resonance imaging (MRI) has demonstrated that NMR and MRI signals associated with a variety of biorelevant imaging agents can be enhanced by many orders of magnitude using a variety of so-called hyperpolarization techniques. This signal enhancement allows for improved spectroscopic analysis of the biorelevant imaging agent as it is metabolized by various tissues at different locations within a body. Analysis of the metabolic information determined by such spectroscopic imaging may allow for non-invasive determination of a health state of tissue within a body. For example, abnormal metabolism of a biorelevant imaging agent may be indicative of a disease such as cancer at some location in the body.

Existing techniques for hyperpolarizing biorelevant imaging agents include dissolution dynamic nuclear polarization (DNP), parahydrogen induced polarization (PHIP), PHIP-sidearm hydrogenation (PHIP-SAH), and signal amplification by reversible exchange (SABRE). In PHIP and PHIP-SAH, a precursor of the biorelevant imaging agent is reacted with parahydrogen to form a parahydrogenated derivative of the precursor. Spin order is then transferred from the protons added via the parahydrogenation reaction to a nucleus of interest (such as a carbon-13 nucleus) contained within the biorelevant imaging agent. In PHIP-SAH, the parahydrogenated derivative of the precursor is cleaved (e.g., hydrolyzed) to yield the hyperpolarized biorelevant imaging agent. The biorelevant imaging agent is then purified and used in an NMR or MRI procedure. In PHIP-SAH, the precursor can comprise a biorelevant imaging agent coupled to a sidearm containing at least one unsaturated bond (e.g., at least one carbon-carbon double bond or at least one carbon-carbon triple bond) suitable for reaction with parahydrogen. However, previous precursors have used sidearms that may not allow the generation of biorelevant imaging agents with clinically relevant polarizations, concentrations, volumes, or purities. Such behavior may be related to poor solubility of the precursors in organic solvents (where parahydrogen is highly soluble), poor yields in the reaction between the unsaturated bond and parahydrogen, poor transfer of spin order (e.g., from the sidearm to the nucleus of interest), or a variety of other factors. Accordingly, there is a need for new PHIP-SAH precursors that produce hyperpolarized biorelevant imaging agents with clinically relevant polarizations, concentrations, volumes, or purities.

The disclosed embodiments include systems and methods for producing biorelevant imaging agents, in clinically relevant polarizations, concentrations, volumes, and purity. Disclosed embodiments provide technical improvements in polarizing biorelevant imaging agents in solution. These technical improvements support increases in biorelevant imaging agent concentration and the degree of biorelevant polarization.

### Hyperpolarization and Parahydrogen

As used in the present disclosure, hyperpolarization describes a condition in which an absolute value of a difference between a population of spin states (e.g., nuclear spin states, proton spin states, or the like) being in one state (e.g., spin up) and a population of a spin states being in another state (e.g., spin down) exceeds the absolute value of the corresponding difference at thermal equilibrium.

Parahydrogen can be used as a source of polarization, consistent with disclosed embodiments. Parahydrogen, as described herein, is a form of molecular hydrogen in which the two proton spins are in the singlet state. The disclosed embodiments are not limited to a particular method of generating parahydrogen. Parahydrogen may be formed in a gas form or in a liquid form. In some embodiments, parahydrogen is generated in gas form by flowing hydrogen gas at low temperature through a chamber with a catalyst (e.g., iron oxide or another suitable catalyst). The hydrogen gas can contain both parahydrogen and orthohydrogen. The low temperature can bring the hydrogen gas to thermodynamic equilibrium in the chamber, increasing the population of parahydrogen.

The disclosed embodiments are not limited to a particular parahydrogen generation location or use location. Parahydrogen can be generated at a first location and subsequently transported to a second location for use. In some embodiments, the first location is a chamber, which may be part of a container, bottle, holder or other regions capable of holding a gas or a liquid. Such a chamber may be maintained at a suitable pressure or temperature. In some embodiments, the first location is a physical location such as a room, a lab, a particular warehouse, hospital or other location where the parahydrogen is generated.

The disclosed embodiments are not limited to a particular parahydrogen transport method. The generated parahydrogen may be transported in a chamber, which may be different from the chamber where the parahydrogen was generated. The chamber transporting the parahydrogen gas may be maintained at a suitable pressure or temperature, which may be transported by vehicle or persons. Transporting the parahydrogen may involve moving the parahydrogen from one container to a different container. Transporting the parahydrogen may involve moving the parahydrogen within the same location, such as from one part of a room to another part of the room. Transporting the parahydrogen may involve moving the parahydrogen from one room in a building to a different room in the same building or to a nearby building. Transporting the parahydrogen may involve moving the parahydrogen to a different location in another part of the same city, or a different city. Transporting the parahydrogen may involve bringing the parahydrogen into the vicinity of a polarizer, an NMR device, or an MRI device. Transporting the parahydrogen may involve packaging or shipping the parahydrogen in suitable containers.

In some embodiments, a population difference between two spin states is the difference between the population of the two spin states divided by the total population of the two spin states. A population difference may be expressed as a fractional population difference or a percentage population difference. In some embodiments, the fractional population difference is at least about 0.1, 0.2, 0.3, 0.4, 0.5, 0.6, 0.7, 0.8, 0.9, or more, at most about 0.9, 0.8, 0.7, 0.6, 0.5, 0.4, 0.3, 0.2, 0.1, or less, or within a range defined by any two of the preceding values.

Hydrogen gas can exhibit a population difference between proton spin states which greatly exceeds the population difference between proton spin states at thermal equilibrium. Parahydrogen can have a large population difference between the singlet spin state and any of the triplet spin states. In the case of Iz1Iz2, there is a large population difference, for example, between the spin state |↑>|↓> and the spin state |↑>|↑>. The population difference in proton spin states can be at least about 0.1 (e.g., a 10% difference in spin states −55% of the parahydrogen molecules in a sample being in the singlet state and 45% in the triplet state), 0.2, 0.3, 0.4, 0.5, 0.6, 0.7, 0.8, 0.9, or more, at most about 0.9, 0.8, 0.7, 0.6, 0.5, 0.4, 0.3, 0.2, 0.1, or less, or within a range defined by any two of the preceding values.

### Biorelevant Imaging Agents

The disclosed embodiments include systems and methods for producing and utilizing biorelevant imaging agents with clinically relevant polarizations, concentrations, volumes, or purities. In some embodiments, the method is for preparing an NMR material. In some embodiments, the NMR material is suitable for use in NMR or MRI operations. In some embodiments, the NMR material increases NMR or MRI signal and signal-to-noise ratio (SNR). In some embodiments, the NMR material is suitable for use in solution NMR spectroscopy. In some embodiments, the NMR material is a chemical compound. In some embodiments, the NMR material is a metabolite (e.g., a molecule with a biological relevance such as an amino acid, a saccharide, a derivative thereof, or the like), such as a metabolite suitable for use in an NMR metabolomics application. In some embodiments, the NMR material is suitable for in-vitro probing of the metabolism of a cell culture or other biological tissue. In some embodiments, the NMR material is used in an NMR probe to investigate a transient effect in which high signal enhancement due to hyperpolarization is needed, such as proton exchange between water and biomolecules. In some embodiments, the NMR material is a small molecule or metabolite suitable for injection into a cell, tissue or organism for detection in an MRI scan. In some embodiments, the NMR material is introduced into a chamber for further analysis by NMR or MRI operations. In some embodiments, the NMR material is enriched with one or more deuterium (H) or carbon-13 (¹³C) atoms.

Consistent with disclosed embodiments, NMR material can include biorelevant imaging agents. In some embodiments, the biorelevant imaging agent can be suitable for use in NMR or MRI operations. In some embodiments, the biorelevant imaging agent may increase NMR or MRI signal or signal-to-noise ratio (SNR). In some embodiments, the biorelevant imaging agent can be suitable for use in solution NMR spectroscopy. In some embodiments, the biorelevant imaging agent may be a metabolite (e.g., a molecule with a biological relevance such as an amino acid, a saccharide, a derivative thereof, or the like), such as a metabolite suitable for use in an NMR metabolomics application. In some embodiments the biorelevant imaging agent is used for perfusion imaging or contrast enhanced imaging in MRI scans. In some embodiments, the biorelevant imaging agent is suitable for in-vitro probing of the metabolism of a cell culture or other biological tissue. In some embodiments, the biorelevant imaging agent is used for in-vitro probing of the metabolism of a cell culture or other biological tissue. In some embodiments, the biorelevant imaging agent is used in an NMR probe to investigate a transient effect in which high signal enhancement due to hyperpolarization is needed, such as proton exchange between water and biomolecules. In some embodiments, the biorelevant imaging agent is a small molecule or metabolite suitable for injection into a cell, tissue or organism for detection in an MRI scan. In some embodiments, the biorelevant imaging agent is introduced into a chamber for further analysis by NMR or MRI operations. In some embodiments, the biorelevant imaging agent is enriched with one or more ²H or ¹³C atoms.

In some embodiments, the biorelevant imaging agent comprises pyruvate, lactate, alpha-ketoglutarate, bicarbonate, fumarate, urea, dehydroascorbate, glutamate, glutamine, acetate, dihydroxyacetone, acetoacetate, glucose, ascorbate, zymonate, alanine, fructose, imidazole, nicotinamide, nitroimidazole, pyrazinamide, isoniazid, a conjugate acid of any of the foregoing, natural and unnatural amino acids, esters thereof, or ²H, ¹³C, or nitrogen-15 (¹⁵N) enriched versions of any of the foregoing. In some embodiments, the biorelevant imaging agent comprises pyruvate, lactate, alpha-ketoglutarate. In some embodiments, the biorelevant imaging agent comprises pyruvate. In some embodiments, the biorelevant imaging agent comprises lactate. In some embodiments, the biorelevant imaging agent comprises alpha-ketoglutarate (e.g., ethyl alpha-ketoglutarate).

In some embodiments, the biorelevant imaging agent comprises at least one non-hydrogen nuclear spin. In some embodiments, the non-hydrogen nuclear comprises at least one spin-½ atom. In some embodiments, the non-hydrogen nuclear spin comprises ¹³C or ¹⁵N. In some embodiments, the biorelevant imaging agent is at least partially isotopically labeled with the non-hydrogen nuclear spin. In some embodiments, the biorelevant imaging agent is at least partially enriched with the non-hydrogen nuclear spin when compared to an analog of the biorelevant imaging agent that features the non-hydrogen nuclear spin at its natural abundance. In some embodiments, the biorelevant imaging agent is enriched to feature the non-hydrogen nuclear spin at an abundance of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more, at most about 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less, or an abundance that is within a range defined by any two of the preceding values.

In some embodiments, the non-hydrogen nuclear spin replaces an NMR-inactive (i.e., spin-0) nucleus (e.g., ¹²C or a quadrupolar (i.e., spin >½) nucleus (e.g., nitrogen-14, ¹⁴N) of the analog of the biorelevant imaging agent that features the non-hydrogen nuclear spin at its natural abundance. For example, an analog of pyruvate that features ¹³C at its natural abundance may include about 98.9% ¹²C and about 1.1% ¹³C at either C* in the structure H₃C—C*(═O)—C*OOH. As a biorelevant imaging agent, pyruvate may instead be isotopically enriched with ¹³C such that one or both C* comprises ¹³C at any abundance described herein. As used herein, *C and C* describe a carbon that can be either a ²C or ¹³C carbon isotope. As another example, an analog of urea that features ¹⁵N at its natural abundance may include about 99.6% ¹⁴N and about 0.4% ¹⁵N at either N* in the structure H₂N*—C(═O)—*NH₂. As a biorelevant imaging agent, urea may instead be isotopically enriched with ¹⁵N such that one or both N* comprises ¹⁵N at any abundance described herein. As used herein, *N and N* describe a nitrogen that can be either a 14N or ¹⁵N nitrogen isotope.

### Biorelevant Imaging Agent Precursors

In some embodiments, the present disclosure describes precursors (i.e., precursor compounds) which comprise a biorelevant imaging agent and a sidearm. In some embodiments, the biorelevant imaging agent is covalently attached to the sidearm. In some embodiments, the biorelevant imaging agent is attached to the sidearm through a transfer moiety, such as a PHIP transfer moiety, which is part of the sidearm.

In some embodiments, the present disclosure describes precursors (i.e., precursor compounds) which comprise an acyl derivative of a biorelevant imaging agent (i.e., R—C(═O)—) and a sidearm. As used herein, the term “acyl derivative of a biorelevant imaging agent” refers to a covalently-bonded derivative of a biorelevant imaging agent in which a terminal acid moiety [R—C(═O)OH)] of an unbonded biorelevant imaging agent is altered to an acyl group and covalent bond [R—C(═O)—)] in the bonded biorelevant imaging agent. In some embodiments, the acyl derivative of the biorelevant imaging agent is covalently attached to the sidearm. In some embodiments, the acyl derivative of the biorelevant imaging agent is attached to the sidearm through a transfer moiety, such as a PHIP transfer moiety, which is part of the sidearm.

The sidearm can be parahydrogenated using parahydrogen (e.g., by mixing the precursor and the parahydrogen). In some embodiments, the hydrogenation creates Iz1Iz2 order, the lower energy state between |↑>|↓>, |↓>|↑> or singlet spin order on two hydrogens spins, depending on whether the hydrogenation is performed at a low magnetic field or high magnetic field.

In some embodiments, the precursor is chosen such that, following hydrogenation and other optional chemical reactions, the biorelevant imaging agent is suitable for use in hyperpolarized NMR or MRI applications. In some embodiments, additional chemical reactions following hydrogenation can be used to separate the biorelevant imaging agent from the precursor. Such additional chemical reactions may include cleaving the sidearm of the precursor, e.g., by hydrolysis. For example, the biorelevant imaging agent can be a metabolite molecule, such that the precursor can a derivative of the metabolite molecule, with the derivative having the generic chemical structure of Formula Ia or Formula Ib. The biorelevant imaging agent can be polarized using the PHIP-SAH method (i.e., parahydrogenation of the sidearm and subsequent polarization transfer to the biorelevant imaging agent). Following hydrogenation and polarization transfer, the linking bond in the precursor (e.g., ester bond) may be hydrolyzed to produce a polarized biorelevant imaging agent and a separate sidearm element.

As used herein, hydrolysis is defined as the cleavage of a molecule via a nucleophilic substitution reaction, with the addition of the elements of water. Hydrolysis can also be performed under anhydrous conditions in the presence of hydroxide ions.

Consistent with disclosed embodiments, precursors of the general chemical form presented in Formula Ia or Formula Ib can be used as precursors for PHIP-SAH. Following hydrogenation of such precursors, the two ¹H spins exhibiting the spin order are near (e.g., only three, four, or five bonds away) from the target carbon or nitrogen on the metabolite, which can be ¹³C enriched or ¹⁵N enriched as described herein. In some embodiments, a high J-coupling between the ¹³C or ¹⁵N spin and at least one of the ¹H spins derived from parahydrogen is achieved. In some embodiments, a J-coupling is achieved of at least about 0.1 hertz (Hz), 0.2 Hz, 0.3 Hz, 0.4 Hz, 0.5 Hz, 0.6 Hz, 0.7 Hz, 0.8 Hz, 0.9 Hz, 1 Hz, 2 Hz, 3 Hz, 4 Hz, 5 Hz, 6 Hz, 7 Hz, 8 Hz, 9 Hz, 10 Hz, or more, at most about 10 Hz, 9 Hz, 8 Hz, 7 Hz, 6 Hz, 5 Hz, 4 Hz, 3 Hz, 2 Hz, 1 Hz, 0.9 Hz, 0.8 Hz, 0.7 Hz, 0.6 Hz, 0.5 Hz, 0.4 Hz, 0.3 Hz, 0.2 Hz, 0.1 Hz, or less, or a J-coupling that is within a range defined by any two of the preceding values. For instance, in some embodiments, the J-coupling is between 1 Hz and 2 Hz, between 1 Hz and 3 Hz, between 1 Hz and 4 Hz, between 1 Hz and 5 Hz, between 1 Hz and 6 Hz, between 1 Hz and 7 Hz, between 1 Hz and 8 Hz, between 1 Hz and 9 Hz, between 1 Hz and 10 Hz, between 2 Hz and 3 Hz, between 2 Hz and 4 Hz, between 2 Hz and 5 Hz, between 2 Hz and 6 Hz, between 2 Hz and 7 Hz, between 2 Hz and 8 Hz, between 2 Hz and 9 Hz, between 2 Hz and 10 Hz, between 3 Hz and 4 Hz, between 3 Hz and 5 Hz, between 3 Hz and 6 Hz, between 3 Hz and 7 Hz, between 3 Hz and 8 Hz, between 3 Hz and 9 Hz, between 3 Hz and 10 Hz, between 4 Hz and 5 Hz, between 4 Hz and 6 Hz, between 4 Hz and 7 Hz, between 4 Hz and 8 Hz, between 4 Hz and 9 Hz, between 4 Hz and 10 Hz, between 5 Hz and 6 Hz, between 5 Hz and 7 Hz, between 5 Hz and 8 Hz, between 5 Hz and 9 Hz, between 5 Hz and 10 Hz, between 6 Hz and 7 Hz, between 6 Hz and 8 Hz, between 6 Hz and 9 Hz, between 6 Hz and 10 Hz, between 7 Hz and 8 Hz, between 7 Hz and 9 Hz, between 7 Hz and 10 Hz, between 8 Hz and 9 Hz, between 8 Hz and 10 Hz, or between 9 Hz and 10 Hz. Such a J-coupling may enable efficient polarization of the ¹³C spin.

Disclosed herein are novel precursors, including the compounds of Formulas Ia, Ib, IIa, IIb, IIIa, IIIb, and IVa, tautomers thereof, deuterated derivatives of those compounds and their tautomers, salts thereof, and ¹³C or ¹⁵N enriched derivatives at one or more sites within the molecule (which may be in turn subject to hyperpolarization), and the subsequent generation of precursors given by the general Formulas Ia, Ib, IIa, IIb, IIIa, IIIb, and IVa.

### Precursors of Formula Ia and Formula Ib

In some embodiments, the precursor comprises a compound of Formula Ia. Formula Ia encompasses the following structure:

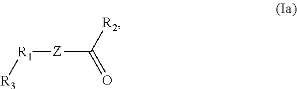

and includes tautomers thereof, deuterated derivatives of those compounds and their tautomers, pharmaceutically acceptable salts thereof, and ¹³C or ¹⁵N enriched derivatives at one or more sites. In some embodiments, Z of Formula Ia describes: (i) a carbon-carbon double bond (—C═C—) which is fully substituted to include ²H (deuterium, also referred to as D) (i.e., —CD=CD-) or (ii) a carbon-carbon triple bond (—C═C—). In some embodiments, R₁ of Formula Ia comprises a parahydrogen induced polarization (PHIP) transfer moiety, as described herein. In some embodiments, R₂ of Formula Ia comprises an optionally substituted hydrocarbon, alkoxy group, primary amine, secondary amine, or tertiary amine, as described herein. In some embodiments, R₃ of Formula Ia comprises a biorelevant imaging agent, as described herein. In Formula Ia, all moieties to the right of the R₃-R₁ bond (i.e., —R₁—Z—(C═O)—R₂) may be collectively referred to as a sidearm.

In some embodiments, the precursor comprises a compound of Formula Ib. Formula Ib encompasses the following structure:

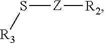

and includes tautomers thereof, deuterated derivatives of those compounds and their tautomers, pharmaceutically acceptable salts thereof, and ¹³C enriched derivatives at one or more sites. In some embodiments, Z in Formula Ib denotes an ethynyl (—C≡C—) group, a fully deuterated prop-2-ynyl (—CD₂-C≡CD₂-) group, a fully deuterated ethenyl (—CD=CD-) group, a fully deuterated prop-2-enyl (—CD₂-CD=CD-) group, or a fully deuterated but-3-enyl (—CD₂-CD₂-CD=CD-) group. In some embodiments, R₂ in Formula Ib comprises an optionally substituted hydrocarbon group, alkyl group, cyclic alkyl group, aryl group, carboxyl group, keto group, or alkoxy group, as described herein. In some embodiments, R₃ in Formula Ib comprises an acyl derivative of a biorelevant imaging agent, as described herein. In Formula Ib, all moieties to the right of the R₃ group (i.e., —S—Z—R₂) may be collectively referred to as a sidearm.

In some embodiments, the compound of Formula Ia or Formula Ib has a solubility in water of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in water that is within a range defined by any two of the preceding values.

In some embodiments, the compound of Formula Ia or Formula Ib has a solubility in an organic solvent (e.g., acetone, ethanol, chloroform, toluene) of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in an organic solvent that is within a range defined by any two of the preceding values.

### Parahydrogenated Precursors of Formula IIa and Formula IIb

In some embodiments, the compound of Formula Ia is parahydrogenated (i.e., modified via the addition of parahydrogen protons across Z via a hydrogenation reaction between Formula Ia and a parahydrogen molecule), as described herein. In some embodiments, parahydrogenation of a compound of Formula Ia yields a compound of Formula IIa. Formula IIa encompasses the following structure:

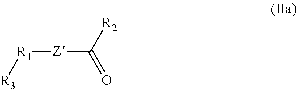

and includes tautomers thereof, deuterated derivatives of those compounds and their tautomers, pharmaceutically acceptable salts thereof, and ¹³C or ¹⁵N enriched derivatives at one or more sites. In some embodiments, Z′ of Formula IIa is: (i) a parahydrogenated carbon-carbon single bond (—CH*—CH*—) which is fully substituted to include ²H (deuterium, also referred to as D) (i.e., —CDH*—CDH*—), or (ii) a parahydrogenated carbon-carbon double bond (—CH*═CH*—). In some embodiments, H* denotes a hydrogen having a spin order derived from parahydrogen (i.e., a hydrogen atom or proton added across the carbon-carbon double bond or the carbon-carbon triple bond Z via a hydrogenation reaction between a compound of Formula Ia and a parahydrogen, as described herein). In some embodiments, H* denotes a hydrogen having a spin order derived from parahydrogen (e.g., before polarization transfer). In some embodiments, R₁ of Formula IIa comprises a PHIP transfer moiety, as described herein. In some embodiments, R₂ of Formula IIa comprises an optionally substituted hydrocarbon, alkoxy group, primary amine, secondary amine, or tertiary amine, as described herein. In some embodiments, R₃ of Formula IIa comprises a biorelevant imaging agent, as described herein. In Formula IIa, all moieties to the right of the R₃—R₁ bond (i.e., —R₁—Z′—(C═O)—R₂) may be collectively referred to as a parahydrogenated sidearm.

In some embodiments, the compound of Formula Ib is parahydrogenated (i.e., modified via the addition of parahydrogen protons across Z via a hydrogenation reaction between Formula Ib and a parahydrogen molecule), as described herein. In some embodiments, parahydrogenation of a compound of Formula Ib yields a compound of Formula IIb. Formula IIb encompasses the following structure:

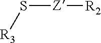

and includes tautomers thereof, deuterated derivatives of those compounds and their tautomers, pharmaceutically acceptable salts thereof, and ¹³C enriched derivatives at one or more sites. In some embodiments, Z′ of Formula IIb denotes a parahydrogenated ethenyl (—CH*═CH*—) group, a fully deuterated parahydrogenated prop-2-enyl (—CD₂-CH*═CH*—) group, a fully deuterated parahydrogenated ethanyl (—CDH*—CDH*—) group, a fully deuterated parahydrogenated propanyl (—CD₂-CDH*—CDH*—) group, or a fully deuterated parahydrogenated butanyl (—CD₂-CD₂-CH*═CH*—) group. In some embodiments, H* denotes a hydrogen having a spin order derived from parahydrogen (i.e., a hydrogen atom or proton added across the carbon-carbon double bond or the carbon-carbon triple bond Z via a hydrogenation reaction between a compound of Formula Ib and a parahydrogen, as described herein). In some embodiments, H* denotes a hydrogen having a spin order derived from parahydrogen (e.g., before polarization transfer). In some embodiments, R₂ of Formula IIb comprises an optionally substituted hydrocarbon group, alkyl group, cyclic alkyl group, aryl group, carboxyl group, keto group, or alkoxy group, as described herein. In some embodiments, R₃ of Formula IIb comprises an acyl derivative of a biorelevant imaging agent, as described herein. In Formula IIb, all moieties to the right of the R₃ group (i.e., S—Z′—R₂) may be collectively referred to as a parahydrogenated sidearm.

In some embodiments, the compound of Formula IIa or Formula IIb has a solubility in water of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in water that is within a range defined by any two of the preceding values.

In some embodiments, the compound of Formula IIa or Formula IIb has a solubility in an organic solvent (e.g., acetone, ethanol, chloroform, toluene) of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in an organic solvent that is within a range defined by any two of the preceding values.

In some embodiments, when the composition of Formula Ia or Formula IIb is reacted with parahydrogen, the chemical yield (e.g., chemical yield of a compound of Formula IIa or Formula IIb) is at least about 30%, 35%, 40%, 45%, 50%, 55%, 60%, 65%, 70%, 75%, 80%, 85%, 90%, 95%, or more, at most about 95%, 90%, 85%, 80%, 75%, 70%, 65%, 60%, 55%, 50%, 45%, 40%, 35%, 30%, or less, or within a range defined by any two of the preceding values. For instance, in some embodiments, when the composition of Formula Ia or Formula Ib is reacted with parahydrogen, the chemical yield is between 30% and 35%, between 30% and 40%, between 30% and 45%, between 30% and 50%, between 30% and 55%, between 30% and 60%, between 30% and 65%, between 30% and 70%, between 30% and 75%, between 30% and 80%, between 30% and 85%, between 30% and 90%, between 30% and 95%, between 35% and 40%, between 35% and 45%, between 35% and 50%, between 35% and 55%, between 35% and 60%, between 35% and 65%, between 35% and 70%, between 35% and 75%, between 35% and 80%, between 35% and 85%, between 35% and 90%, between 35% and 95%, between 40% and 45%, between 40% and 50%, between 40% and 55%, between 40% and 60%, between 40% and 65%, between 40% and 70%, between 40% and 75%, between 40% and 80%, between 40% and 85%, between 40% and 90%, between 40% and 95%, between 45% and 50%, between 45% and 55%, between 45% and 60%, between 45% and 65%, between 45% and 70%, between 45% and 75%, between 45% and 80%, between 45% and 85%, between 45% and 90%, between 45% and 95%, between 50% and 55%, between 50% and 60%, between 50% and 65%, between 50% and 70%, between 50% and 75%, between 50% and 80%, between 50% and 85%, between 50% and 90%, between 50% and 95%, between 55% and 60%, between 55% and 65%, between 55% and 70%, between 55% and 75%, between 55% and 80%, between 55% and 85%, between 55% and 90%, between 55% and 95%, between 60% and 65%, between 60% and 70%, between 60% and 75%, between 60% and 80%, between 60% and 85%, between 60% and 90%, between 60% and 95%, between 65% and 70%, between 65% and 75%, between 65% and 80%, between 65% and 85%, between 65% and 90%, between 65% and 95%, between 70% and 75%, between 70% and 80%, between 70% and 85%, between 70% and 90%, between 70% and 95%, between 75% and 80%, between 75% and 85%, between 75% and 90%, between 75% and 95%, between 80% and 85%, between 80% and 90%, between 80% and 95%, between 85% and 90%, between 85% and 95%, or between 90% and 95%.

### Cleaved Precursors of Formula IIIa and Formula IIIb

In some embodiments, a compound of Formula IIa is cleaved (e.g., hydrolyzed), as described herein. In some embodiments, a compound of Formula IIa is cleaved (e.g., hydrolyzed), as described herein, to provide a sidearm compound and a corresponding biorelevant imaging agent. In some embodiments, cleavage of a compound of Formula IIa yields a compound of Formula IIIa and a corresponding biorelevant imaging agent, as described herein. Formula IIIa encompasses the following structure:

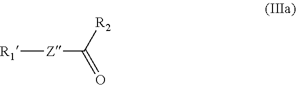

and includes tautomers thereof, deuterated derivatives of those compounds and their tautomers, pharmaceutically acceptable salts thereof, and ¹³C or ¹⁵N enriched derivatives at one or more sites. In some embodiments, Z″ of Formula IIIa is: (i) a parahydrogenated carbon-carbon single bond (—CH*—CH*—) which is fully substituted to include ²H (deuterium, also referred to as D) (i.e., —CDH*—CDH*—), or (ii) a parahydrogenated carbon-carbon double bond (—CH*═CH*—). In some embodiments, R₁′ of Formula IIIa comprises a PHIP transfer moiety, as described herein. In some embodiments, R₂ of Formula IIIa comprises an optionally substituted hydrocarbon, alkoxy group, primary amine, secondary amine, or tertiary amine, as described herein. In Formula IIIa, all of moieties R₁—Z″—(C═O)—R₂ may be collectively referred to as a cleaved sidearm or a hydrolyzed sidearm.

In some embodiments, a compound of Formula IIb is cleaved (e.g., hydrolyzed), as described herein. In some embodiments, a compound of Formula IIb is cleaved (e.g., hydrolyzed), as described herein, to provide a sidearm compound and a corresponding biorelevant imaging agent. In some embodiments, cleavage of a compound of Formula IIb yields a compound of Formula IIIb and a corresponding biorelevant imaging agent, as described herein. Formula IIIb encompasses the following structure:

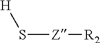

and includes tautomers thereof, deuterated derivatives of those compounds and their tautomers, pharmaceutically acceptable salts thereof, and ¹³C enriched derivatives at one or more sites. In some embodiments, Z″ of Formula IIIb denotes a parahydrogenated ethenyl (—CH*═CH*—) group, a fully deuterated parahydrogenated prop-2-enyl (—CD₂-CH*═CH*—) group, a fully deuterated parahydrogenated ethanyl (—CDH*—CDH*—) group, a fully deuterated parahydrogenated propanyl (—CD₂-CDH*—CDH*—) group, or a fully deuterated parahydrogenated butanyl (—CD₂-CD₂-CH*═CH*—) group. R₂ of Formula IIIb comprises an optionally substituted hydrocarbon group, alkyl group, cyclic alkyl group, aryl group, carboxyl group, keto group, or alkoxy group, as described herein. In Formula IIIb, all of moieties H—S—Z′—R₂ may be collectively referred to as a cleaved sidearm or a hydrolyzed sidearm.

In some embodiments, the compound of Formula IIIa or Formula IIIb has a solubility in water of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in water that is within a range defined by any two of the preceding values.

In some embodiments, the compound of Formula IIIa or Formula IIIb has a solubility in an organic solvent (e.g., acetone, ethanol, chloroform, toluene) of at least about 1 millimolar (mM), 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, 550 mM, 600 mM, 650 mM, 700 mM, 750 mM, 800 mM, 850 mM, 900 mM, 950 mM, 1,000 mM, or more, at most about 1,000 mM, 950 mM, 900 mM, 850 mM, 800 mM, 750 mM, 700 mM, 650 mM, 600 mM, 550 mM, 500 mM, 450 mM, 400 mM, 350 mM, 300 mM, 250 mM, 200 mM, 150 mM, 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, or less, or a solubility in an organic solvent that is within a range defined by any two of the preceding values.

### Sidearms of Formula IVa

In some embodiments, biorelevant imaging agents and sidearms, such as the sidearm compound of Formula IVa, are conjugated to form precursor compounds, such as the compound of Formula Ia, as described herein. Formula IVa encompasses the following structure:

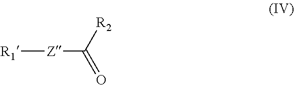

and includes tautomers thereof, deuterated derivatives of those compounds and their tautomers, pharmaceutically acceptable salts thereof, and ¹³C or ¹⁵N enriched derivatives at one or more sites. In some embodiments, Z of Formula IVa describes: (i) a carbon-carbon double bond (—C═C—) which is fully substituted to include ²H (deuterium, also referred to as D) (i.e., —CD=CD-) or (ii) a carbon-carbon triple bond (—C≡C—). In some embodiments, R₁ of Formula IVa comprises a parahydrogen induced polarization (PHIP) transfer moiety, as described herein. In some embodiments, R₂ of Formula IVa comprises a solubilizing moiety, as described herein. In some embodiments, R₂ of Formula IVa comprises an optionally substituted hydrocarbon, alkoxy group, primary amine, secondary amine, or tertiary amine. In some embodiments, conjugation of a compound of Formula IVa with a biorelevant imaging agent yields a compound of Formula Ia, as described herein.

### PHIP Transfer Moieties

In some embodiments, compositions of the present disclosure comprise a PHIP transfer moiety. In some embodiments, compositions of the present disclosure comprise a PHIP transfer moiety between the Z, Z′, or Z″ moiety and the sulfur atom of Formula Ib, Formula IIb, or Formula IIIb. In some embodiments, a PHIP transfer moiety described herein comprises a chemical moiety configured to permit or enhance polarization transfer from one or more parahydrogenated protons H* (e.g., H* in a sidearm) to one or more non-hydrogen nuclear spins of a biorelevant imaging agent (such as one or more ³C or ¹⁵N atoms of a biorelevant imaging agent, as described herein). In some embodiments, the PHIP transfer moiety permits or enhances polarization transfer from the parahydrogen protons H* in the sidearm of the compound of Formula IIa or Formula IIb to the non-hydrogen nuclear spins of the corresponding biorelevant imaging agent of the compound of Formula IIa or Formula IIb. In some embodiments, the PHIP transfer moiety permits or enhances polarization transfer from the parahydrogen protons H* in the sidearm of the compound of Formula IIa or Formula IIb to the non-hydrogen nuclear spins of the corresponding biorelevant imaging agent of the compound of Formula IIa or Formula IIb, following the parahydrogenation reaction between parahydrogen and Formula Ia or Formula Ib.

In some embodiments, the PHIP transfer moiety comprises a fully deuterated C1 hydrocarbon (i.e., —CD₂-) or a fully deuterated C2 hydrocarbon (i.e., —CD₂-CD₂-).

In some embodiments, the PHIP transfer moiety comprises a chemical moiety of the form *CR₄R₅ or any fully deuterated version thereof. In some embodiments, *C is a ¹²C carbon isotope. In some embodiments, R₄ and R₅ are each independently selected from: ²H, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

In some embodiments, the PHIP transfer moiety comprises a chemical moiety of the form *CR₆R₇—*CR₈R₉ or any fully deuterated version thereof. In some embodiments, *C is a ¹²C carbon isotope. In some embodiments, R₆, R₇, R₈, and R₉ are each independently selected from: ²H, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

In some embodiments, the PHIP transfer moiety comprises a chemical moiety of the form *CH₂, *CH₂—*CH₂, or any fully deuterated version thereof. In some embodiments, *C is a ¹²C carbon isotope.

In some embodiments, the compositions described herein comprise a first J-coupling J₁₂ between a spin-½ atom described herein and a non-hydrogen nuclear spin described herein. In some embodiments, the compositions described herein comprise a second J-coupling J₁₃ between the spin-½ atom described herein and parahydrogen protons H* described herein. In some embodiments, the compositions described herein comprise a third J-coupling J₂₃ between the non-hydrogen nuclear spin described herein and the parahydrogen protons H* described herein. In some embodiments, J₁₂ and/or J₁₃ is greater than J₂₃. In such cases, the PHIP transfer moiety may permit or enhance polarization transfer.

In some embodiments, the PHIP transfer moiety induces a J-coupling between one or both of the *H nuclear spins with the non-hydrogen nuclear spins of at least about 0.1 Hz, 0.2 Hz, 0.3 Hz, 0.4 Hz, 0.5 Hz, 0.6 Hz, 0.7 Hz, 0.8 Hz, 0.9 Hz, 1 Hz, or more, at most about 1 Hz, 0.9 Hz, 0.8 Hz, 0.7 Hz, 0.6 Hz, 0.5 Hz, 0.4 Hz, 0.3 Hz, 0.2 Hz, 0.1 Hz, or less, or a J-coupling with the non-hydrogen nuclear spins that is within a range defined by any two of the preceding values. For instance, in some embodiments, the J-coupling is between 0.1 Hz and 0.2 Hz, between 0.1 Hz and 0.3 Hz, between 0.1 Hz and 0.4 Hz, between 0.1 Hz and 0.5 Hz, between 0.1 Hz and 0.6 Hz, between 0.1 Hz and 0.7 Hz, between 0.1 Hz and 0.8 Hz, between 0.1 Hz and 0.9 Hz, between 0.1 Hz and 1 Hz, between 0.2 Hz and 0.3 Hz, between 0.2 Hz and 0.4 Hz, between 0.2 Hz and 0.5 Hz, between 0.2 Hz and 0.6 Hz, between 0.2 Hz and 0.7 Hz, between 0.2 Hz and 0.8 Hz, between 0.2 Hz and 0.9 Hz, between 0.2 Hz and 1 Hz, between 0.3 Hz and 0.4 Hz, between 0.3 Hz and 0.5 Hz, between 0.3 Hz and 0.6 Hz, between 0.3 Hz and 0.7 Hz, between 0.3 Hz and 0.8 Hz, between 0.3 Hz and 0.9 Hz, between 0.3 Hz and 1 Hz, between 0.4 Hz and 0.5 Hz, between 0.4 Hz and 0.6 Hz, between 0.4 Hz and 0.7 Hz, between 0.4 Hz and 0.8 Hz, between 0.4 Hz and 0.9 Hz, between 0.4 Hz and 1 Hz, between 0.5 Hz and 0.6 Hz, between 0.5 Hz and 0.7 Hz, between 0.5 Hz and 0.8 Hz, between 0.5 Hz and 0.9 Hz, between 0.5 Hz and 1 Hz, between 0.6 Hz and 0.7 Hz, between 0.6 Hz and 0.8 Hz, between 0.6 Hz and 0.9 Hz, between 0.6 Hz and 1 Hz, between 0.7 Hz and 0.8 Hz, between 0.7 Hz and 0.9 Hz, between 0.7 Hz and 1 Hz, between 0.8 Hz and 0.9 Hz, between 0.8 Hz and 1 Hz, or between 0.9 Hz and 1 Hz.

### Deuterated Compounds

In some embodiments, using deuterated Z, Z′, or Z″ groups, or PHIP transfer moieties increases the period of time for which spin order associated with the parahydrogen singlet state (referred to herein as “singlet lifetime”) persists. In some embodiments, the singlet lifetime associated with a compound containing deuterated Z, Z′, or Z″ groups, or PHIP transfer moieties, is increased when compared with a similar compound containing Z, Z′, or Z″ groups, or PHIP transfer moieties, that contain nuclei such as ¹H, ¹³C, ¹⁹F, ³¹P, or other nuclei that couple to the H* hydrogen atoms having spin order derived from parahydrogen described herein. In some embodiments, the singlet lifetime associated with compounds containing the deuterated Z, Z′, or Z″ groups, or PHIP transfer moieties described herein is at least about 10 seconds, 15 seconds, 20 seconds, 25 seconds, 30 seconds, 35 seconds, 40 seconds, 45 seconds, 50 seconds, 55 seconds, 60 seconds, 65 seconds, 70 seconds, 75 seconds, 80 seconds, 85 seconds, 90 seconds, 95 seconds, 100 seconds, 105 seconds, 110 seconds, 115 seconds, 120 seconds, or more, at most about 120 seconds, 115 seconds, 110 seconds, 105 seconds, 100 seconds, 95 seconds, 90 seconds, 85 seconds, 80 seconds, 75 seconds, 70 seconds, 65 seconds, 60 seconds, 55 seconds, 50 seconds, 45 seconds, 40 seconds, 35 seconds, 30 seconds, 25 seconds, 20 seconds, 15 seconds, 10 seconds, or less, or within a range defined by any two of the preceding values.

In some embodiments, the Z, Z′, or Z″ groups, or PHIP transfer moieties contain at most 2, 1, or 0 nuclei such as ¹H, ¹³C, ¹⁹F, ³¹P, or other nuclei that couple to the H* hydrogen atoms having spin order derived from parahydrogen described herein. In some embodiments, the Z, Z′, or Z″ groups, or PHIP transfer moieties contain at most 2, 1, or 0 ¹H nuclei.

As used herein, the phrase “fully deuterated” refers to the replacement of ¹H (protons) with ²H (deuterium, also referred to as D) at each site in a chemical moiety. Thus, for instance, a fully deuterated linear alkyl hydrocarbon moiety would have the form (—CD₂)ₙ-CD₃, with n ranging from 0 to 9 for a fully deuterated linear C1-C10 alkyl hydrocarbon. Similarly, a fully deuterated phenyl moiety would have the form —C₆D₅, a fully deuterated benzyl moiety would have the form —CD₂-C₆D₅, and so forth.

### R₂ Groups

In some embodiments, an R₂ group described herein comprises an optionally substituted hydrocarbon, alkoxy group, primary amine, secondary amine, or tertiary amine. In some embodiments, an R₂ group described herein comprises an optionally substituted hydrocarbon, alkoxy group, primary amine, secondary amine, or tertiary amine that functions as a solubilizing moiety. In some embodiments, an R₂ group described herein comprises a solubilizing moiety. In some embodiments, the solubilizing moiety comprises any chemical moiety configured to permit or enhance the solubility of a compound, such as any of the compounds of Formula Ia, Ib, IIa, IIb, IIIa, IIIb and/or IVa in a solution in which the parahydrogenation reaction or the cleavage (e.g., hydrolysis) reaction takes place. In some embodiments, the enhancement of the solubility is measured with respect to a variant of the compound of Formula Ia, Ib, IIa, IIb, IIIa, IIIb and/or IVa that utilizes one or more protons in place of the R₂ group. In some embodiments, the enhancement of the solubility is measured with respect to a variant of the compound of Formula Ia, Ib, IIa, IIb, IIIa, IIIb and/or IVa that utilizes a methyl group as the R₂ group.

In some embodiments, the solubilizing moiety comprises a hydrophobic moiety or an organophilic moiety. In some embodiments, the solubilizing moiety comprises an organic solubilizing moiety. For example, in some embodiments, the solubilizing moiety comprises a hydrophobic moiety, an organophilic moiety, or an organic solubilizing moiety. In some embodiments, the solubilizing moiety comprises a hydrophilic moiety or an organophobic moiety.

In some embodiments, the R₂ group comprises, or is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group, a propionic alcohol group, an n-butyl alcohol group, an s-butyl alcohol group, a t-butyl alcohol group, an isobutyl alcohol group, a methoxy group, an ethoxy group, a propoxy group, an isopropoxy group, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, and a tertiary amide group. In some embodiments, the substituted phenyl group is selected from fluorobenzene, chlorobenzene, bromobenzene, iodobenzene, toluene, cumene, ethylbenzene, styrene, ortho-xylene, meta-xylene, para-xylene, phenol, benzoic acid, benzaldehyde, acetophenone, methyl benzoate, anisole, aniline, nitrobenzene, benzonitrile, benzamide, benzenesulfonic acid, naphthalene, and anthracene.

### R₃ Groups

In some embodiments, an R₃ group described herein comprises a biorelevant imaging agent. In some embodiments, the biorelevant imaging agent has the formula R₄C(═O)X—. In some embodiments, R₄ is chosen from a linear, branched, or cyclic C1-C10 alkyl group, in which one or more C atoms are optionally substituted with CO, COOH, CH₂COOH, CONH₂, an OH, an amino (NR′R″), one or more halogen atoms, one or more halo-alkyl groups, or one or more carbocycles, wherein the carbocycle is optionally substituted with one or more aliphatic or aromatic ring, which is optionally substituted by one or more functional groups. In some embodiments, X is chosen from NR′″ and 0. In some embodiments, R′, R″, and R′″ are each independently selected from ¹H, ²H, ³H, and an amino protecting group, optionally selected from trifluoroacetyl, acetyl, benzoyl, carbobenzoxy, tert-butyl carbonate and benzyl. In some embodiments, the R₃ group comprises any biorelevant imaging agent described herein.

In some embodiments, an R₃ group described herein comprises an acyl derivative of a biorelevant imaging agent. In some embodiments, the biorelevant imaging agent has the formula R₉C(═O)O—. In some embodiments, R₉ is chosen from a linear, branched, or cyclic C1-C10 alkyl group, in which one or more C atoms are optionally substituted with CO, COOH, CH₂COOH, CONH₂, an OH, an amino (NR′R″), one or more halogen atoms, one or more halo-alkyl groups, or one or more carbocycles, wherein the carbocycle is optionally substituted with one or more aliphatic or aromatic ring, which is optionally substituted by one or more functional groups. In some embodiments, R′ and R″ are each independently selected from ¹H, ²H, ³H, and an amino protecting group, optionally selected from trifluoroacetyl, acetyl, benzoyl, carbobenzoxy, tert-butyl carbonate and benzyl. In some embodiments, the R₃ group comprises an acyl derivative of any biorelevant imaging agent described herein.

In some embodiments, the R₃ group comprises at least one non-hydrogen nuclear spin. In some embodiments, the non-hydrogen nuclear comprises at least one spin-½ atom. In some embodiments, the non-hydrogen nuclear spin comprises ¹³C or ¹⁵N. In some embodiments, the R₃ group is at least partially isotopically labeled with the non-hydrogen nuclear spin. In some embodiments, the R₃ group is at least partially enriched with the non-hydrogen nuclear spin when compared to an analog of the R₃ group that features the non-hydrogen nuclear spin at its natural abundance. In some embodiments, the R₃ group is enriched to feature the non-hydrogen nuclear spin at an abundance of at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more, at most about 99%, 98%, 97%, 96%, 95%, 94%, 93%, 92%, 91%, 90%, 85%, 80%, 75%, 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less, or an abundance that is within a range defined by any two of the preceding values.

In some embodiments, the non-hydrogen nuclear spin replaces an NMR-inactive (i.e., spin-0) nucleus (e.g., ¹²C or a quadrupolar (i.e., spin >½) nucleus (e.g., ¹⁴N) of the analog of the R₃ group that features the non-hydrogen nuclear spin at its natural abundance, as described herein. In some embodiments, the non-hydrogen nuclear spin is located no more than about 1 or 2 chemical bonds from the carbonyl (C═O) carbon in the R₃ group.

### Parahydrogenation

In some embodiments, the non-hydrogen nuclear spin replaces an NMR-inactive (i.e., spin-0) nucleus (e.g., ¹²C or a quadrupolar (i.e., spin >½) nucleus (e.g., ¹⁴N) of the analog of the R₃ group that features the non-hydrogen nuclear spin at its natural abundance, as described herein. In some embodiments, the non-hydrogen nuclear spin is located no more than about 1 or 2 chemical bonds from the carbonyl (C═O) carbon in the R₃ group.

Consistent with disclosed embodiments, a precursor to the biorelevant imaging agent (such as a compound of Formula Ia or Formula Ib, as described herein) can be parahydrogenated by combining the precursor, parahydrogen, and a hydrogenation catalyst. The disclosed embodiments are not limited to a particular method of generating a parahydrogenated precursor. In some embodiments, the precursor is added to a mixture containing parahydrogen. In some embodiments, parahydrogen gas is added to a solution containing the precursor (e.g., the parahydrogen gas can be bubbled into such a solution). In hydrogenating the precursor, the parahydrogen can create Iz1Iz2 order, preferential population of the lower energy state between |↑>|↓>, |↓>|↑> or singlet spin order on two hydrogens spins in the precursor.

The precursor can have an unsaturated bond (such as an unsaturated carbon-carbon double bond or an unsaturated carbon-carbon triple bond) that can be hydrogenated by the parahydrogen gas. Following combination of the precursor and the parahydrogen, at least about 10%, 20%, 30%, 40%, 50%, 60%, 70%, 80%, 90%, or more of the precursor, at most about 90%, 80%, 70%, 60%, 50%, 40%, 30%, 20%, 10%, or less of the precursor, or a percentage of the precursor that is within a range defined by any two of the preceding values may be hydrogenated.

In some embodiments, the parahydrogenated precursor has a population difference in the parahydrogenated proton spin states of at least about 1%, 2%, 3%, 4%, 5%, 6%, 7%, 8%, 9%, 10%, 15%, 20%, 25%, 30%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1%, or less, or a population difference that is within a range defined by any two of the preceding values. For instance, in some embodiments, the population difference is between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%. In some embodiments, the population difference is between spin states which include the parahydrogenated protons as well as other nuclear spins, for example additional protons on the compound. In some embodiments, the parahydrogenated precursor includes a sidearm and the parahydrogenated spins can be located on the sidearm.

In some embodiments, the concentration of the hydrogenation catalyst during hydrogenation is at least about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, or more, at most about 100 mM, 90 mM, 80 mM, 70 mM, 60 mM, 50 mM, 40 mM, 30 mM, 20 mM, 10 mM, 9 mM, 8 mM, 7 mM, 6 mM, 5 mM, 4 mM, 3 mM, 2 mM, 1 mM, 0.9 mM, 0.8 mM, 0.7 mM, 0.6 mM, 0.5 mM, 0.4 mM, 0.3 mM, 0.2 mM, 0.1 mM, or less, or within a range defined by any two of the preceding values.

The disclosed embodiments can include methods implemented by the disclosed systems for generating a hyperpolarized biorelevant imaging agent. The disclosed methods can include mixing (e.g., by a mixing mechanism) a solution which includes a precursor to the biorelevant imaging agent and a hydrogenation catalyst. A mixing mechanism may be a device for introducing, holding, and facilitating a blend, mixture, or solution of two or more materials. In some embodiments, the mixing mechanism is disposed in a chamber, and the mixing occurs inside the chamber. In some embodiments, the solution is mixed at a location away from the chamber. The solution may be at least about 1 milliliter (ml), 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, or more in volume, at most about 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less in volume, or within a volume range defined by any two of the preceding values.

In some embodiments, the mixing mechanism is a gas-liquid exchange mechanism. For example, the gas-liquid exchange mechanism may be a bubbler or a diffusion system. In some embodiments, the mixing mechanism comprises membranes adapted to permit diffusion of molecular hydrogen. In some embodiments the mixing can be performed using a spray chamber, where the solution is sprayed into a chamber filled with pressurized parahydrogen.

In some embodiments, the catalyst is a molecule, complex or particle system that catalyzes hydrogenation. In some embodiment, the catalyst comprises a homogeneous metal catalyst such as a rhodium complex or a ruthenium complex. The rhodium complex can be used for coordination and activation of precursor molecules and parahydrogen. In some embodiments, a heterogeneous metal catalyst is connected to a nanoparticle.

Various embodiments of the present disclosure describe introducing a solution which includes a precursor to the biorelevant imaging agent and a hydrogenation catalyst into a chamber configured to hold the solution during polarization transfer. In some embodiments, the solution is mixed in the chamber. In some embodiments, the solution is hydrogenated in the chamber. In some embodiments, the chamber is within a magnetic shield (e.g., a mu metal shield). The magnetic shield can reduce the effect of the Earth's magnetic field (or other extraneous magnetic fields), permitting modulation of the amplitude of a low-level magnetic field applied to the solution. Accordingly, placing the solution within the chamber can include placing the solution within the magnetic shield.

As described herein, in some embodiments parahydrogenation occurs prior to polarization transfer (e.g., prior to the modulation of the amplitude the magnetic field applied to the solution, or the like). In some embodiments, parahydrogenation occurs during polarization transfer. For example, parahydrogen can be combined with (e.g., flowed or bubbled through the solution) the solution during modulation of the amplitude of the magnetic field.

In some embodiments, the parahydrogen gas is combined with the solution in a hydrogenation chamber at pressure. The pressure can be at least about 10 bar, 15 bar, 20 bar, 30 bar, 50 bar, or more, at most about 50 bar, 30 bar, 20 bar, 15 bar, 10 bar or less, or within a range defined by any two of the preceding values. In some embodiments, the parahydrogen is combined with the solution in a metallic chamber capable of withstanding the pressure. The parahydrogen can be combined with the solution for (or the dissolution of the parahydrogen can occur in less than) a time interval. The time interval can be at most about 90 seconds, 60 seconds, 30 seconds, 20 seconds, 10 seconds, 9 seconds, 8 seconds, 7 seconds, 6 seconds, 5 seconds, 4 seconds, 3 seconds, 2 seconds, 1 second, or less, at least about 1 second, 2 seconds, 3 seconds, 4 seconds, 5 seconds, 6 seconds, 7 seconds, 8 seconds, 9 seconds, 10 seconds, 20 seconds, 30 seconds, 60 seconds, 90 seconds, or more, or within a range defined by any two of the preceding values. In some embodiments, the hydrogenation is carried out or occurs within the time interval.

### Polarization Transfer Using Radiofrequency Waveforms

In some embodiments, the concentration of the precursor or target molecule in the solution prior to polarization transfer is at least about 10 mM, 20 mM, 30 mM, 40 mM, 50 mM, 60 mM, 70 mM, 80 mM, 90 mM, 100 mM, 200 mM, 300 mM, 400 mM, 500 mM, 600 mM, 700 mM, 800 mM, 900 mM, 1,000 mM, or more, at most about 1,000 mM, 900 mM, 800 mM, 700 mM, 600 mM, 500 mM, 400 mM, 300 mM, 200 mM, 100 mM, 90 mM, 80 mM, 70 mM, 60 mM, 50 mM, 40 mM, 30 mM 20 mM, 10 mM, or less, or within a range defined by any two of the preceding values. The volume of the solution can be at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 200 ml, 300 ml, 400 ml, 500 ml, 600 ml, 700 ml, 800 ml, 900 ml, 1000 ml, 2000 ml, or more, at most about 2000 ml, 1000 ml, 900 ml, 800 ml, 700 ml, 600 ml, 500 ml, 400 ml, 300 ml, 200 ml, 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less, or within a range defined by any two of the preceding values.

Various embodiments of the present disclosure describe applying a polarization transferring magnetic perturbation aimed to generate a magnetic field around the solution (e.g., around a solution containing Formula IIa or Formula IIb described herein). In some embodiments, the magnetic field has a strength of at least about 0.1 gauss (G), 0.2 G, 0.3 G, 0.4 G, 0.5 G, 0.6 G, 0.7 G, 0.8 G, 0.9 G, 1 G, 2 G, 3 G, 4 G, 5 G, 6 G, 7 G, 8 G, 9 G, 10 G, 20 G, 30 G, 40 G, 50 G, 60 G, 70 G, 80 G, 90 G, 100 G, 200 G, 300 G, 400 G, 500 G, 600 G, 700 G, 800 G, 900 G, 1,000 G, 2,000 G, 3,000 G, 4,000 G, 5,000 G, 6,000 G, 7,000 G, 8,000 G, 9,000 G, 10,000 G, 20,000 G, 30,000 G, 40,000 G, 50,000 G, 60,000 G, 70,000 G, 80,000 G, 90,000 G, 100,000 G, 200,000 G, or more, at most about 200,000 G, 100,000 G, 90,000 G, 80,000 G, 70,000 G, 60,000 G, 50,000 G, 40,000 G, 30,000 G, 20,000 G, 10,000 G, 9,000 G, 8,000 G, 7,000 G, 6,000 G, 5,000 G, 4,000 G, 3,000 G, 2,000 G, 1,000 G, 900 G, 800 G, 700 G, 600 G, 500 G. 400 G, 300 G. 200 G, 100 G, 90 G, 80 G, 70 G, 60 G, 50 G, 40 G, 30 G, 20 G, 10 G, 9 G, 8 G, 7 G, 6 G, 5 G, 4 G, 3 G, 2 G, 1 G, 0.9 G, 0.8 G, 0.7 G, 0.6 G, 0.5 G, 0.4 G, 0.3 G, 0.2 G, 0.1 G, or less, or within a range defined by any two of the preceding values. In some embodiments, the magnetic field has a strength of 0.1 G to 200,000 G around the solution.

The magnetic perturbation can be produced by an electro-magnet or a permanent magnet. The magnetic field can be applied to the sample in pulses or in a continuous wave (CW). The magnetic perturbation can be static or time varying.

A signal generator can be configured to generate one or more radiofrequency (RF) waveforms that can be applied to the sample to transfer polarization. The signal generator can include one more computing unit, processors, controllers, associate memories, PCs, computers services, or any devices capable of carrying computational operations using inputs and producing outputs. In some embodiments, RF coils may radiate, or ‘apply’ the pulse sequences, including the first RF waveform. In some embodiments, the RF coils may have one or more channels. Channels may be pathways for RF signals. There may be provided at least one channel for each different type of NMR spectroscopy. In some embodiments, there is at least one channel for ¹H and at least one channel for any of ²H, ¹³C, ¹⁵N, ¹⁹F, and ³¹P. For example, a first RF waveform can be applied to a ¹H channel of the one or more radiofrequency coils (RF coils) disposed around the sample. In some embodiments, a second RF waveform is applied to a ¹³C channel of the RF coils. In some embodiments, the RF waveforms on the ¹H channel and ¹³C channel are configured to apply a polarization transfer sequence, such as PH-INEPT, Goldman's sequence, S2M, S2hM, SLIC, ADAPT or ESOTERIC.

In some embodiments, the RF waveforms is configured to support polarization transfer, even in the presence of a large proton full width half maximum (FWHM). Such RF waveforms can include a pulse sequence, which can include tens to hundreds of RF pulses. The sequence can be configured such that the pulses protect against the detrimental effects of magnetic field inhomogeneities on polarization transfer.

In some embodiments, a pulse sequence for polarization is configured to transfer the spin order from non-equivalent two ¹H hydrogenated spins, e.g., when the chemical shift difference is larger than the J-coupling between them. ESOTHERIC, for example, may be a pulse sequence suited for polarization transfer in this regime.

In some embodiments, the pulse sequence is configured to transfer the spin order from equivalent ¹H hydrogen spins, e.g., when the chemical shift difference is smaller than the J-coupling between them. Such pulse sequences may be used in magnetic fields having a strength of at least about 0.01 millitesla (mT), 0.02 mT, 0.03 mT, 0.04 mT, 0.05 mT, 0.06 mT, 0.07 mT, 0.08 mT, 0.09 mT, 0.1 mT, 0.2 mT, 0.3 mT, 0.4 mT, 0.5 mT, 0.6 mT, 0.7 mT, 0.8 mT, 0.9 mT, 1 mT, 2 mT, 3 mT, 4 mT, 5 mT, 6 mT, 7 mT, 8 mT, 9 mT, 10 mT, 20 mT, 30 mT, 40 mT, 50 mT, 60 mT, 70 mT, 80 mT, 90 mT, 100 mT, 200 mT, 300 mT, 400 mT, 500 mT, 600 mT, 700 mT, 800 mT, 900 mT, 1,000 mT, 2,000 mT, 3,000 mT, 4,000 mT, 5,000 mT, 6,000 mT, or more, at most about 6,000 mT, 5,000 mT, 4,000 mT, 3,000 mT, 2,000 mT, 1,000 mT, 900 mT, 800 mT, 700 mT, 600 mT, 500 mT, 400 mT, 300 mT, 200 mT, 100 mT, 90 mT, 80 mT, 70 mT, 60 mT, 50 mT, 40 mT, 30 mT, 20 mT, 10 mT, 9 mT, 8 mT, 7 mT, 6 mT, 5 mT, 4 mT, 3 mT, 2 mT, 1 mT, 0.9 mT, 0.8 mT, 0.7 mT, 0.6 mT, 0.5 mT, 0.4 mT, 0.3 mT, 0.2 mT, 0.1 mT, 0.09 mT, 0.08 mT, 0.07 mT, 0.06 mT, 0.05 mT, 0.04 mT, 0.03 mT, 0.02 mT, 0.01 mT, or less, or within a range defined by any two of the preceding values. An example of such a sequence may be Goldman's sequence (M. Goldman, H. Jóhannesson, C. R. Phys. 2005, 6, 575-581, which is incorporated herein by reference as related to pulse sequence configurations to transfer spin order), the singlet to heteronuclear magnetization (S2 hM) sequence, or other sequences used in singlet NMR (e.g., ADAPT, SLIC, etc.).

In some embodiments, a magnetic shield is configured to maintain a magnetic field applied to the solution of at least about 0 mG, 0.1 mG, 0.2 mG, 0.3 mG, 0.4 mG, 0.5 mG, 0.6 mG, 0.7 mG, 0.8 mG, 0.9 mG, 1 mG, 2 mG, 3 mG, 4 mG, 5 mG, 6 mG, 7 mG, 8 mG, 9 mG, 10 mG, 20 mG, 30 mG, 40 mG, 50 mG, 60 mG, 70 mG, 80 mG, 90 mG, 100 mG, or more, at most about 100 mG, 90 mG, 80 mG, 70 mG, 60 mG, 50 mG, 40 mG, 30 mG, 20 mG, 10 mG, 9 mG, 8 mG, 7 mG, 6 mG, 5 mG, 4 mG, 3 mG, 2 mG, 1 mG, 0.9 mG, 0.8 mG, 0.7 mG, 0.6 mG, 0.5 mG, 0.4 mG, 0.3 mG, 0.2 mG, 0.1 mG or less, or a magnetic field that is within a range defined by any two of the preceding values. The magnetic shield can maintain the magnetic field strength within the polarization chamber at such amplitudes during application of the polarization waveform to the one or more radiofrequency coils.

Consistent with disclosed embodiments, the RF waveform can be applied to a solution containing a parahydrogenated precursor.

### Transferring Polarization Using Magnetic Field Modulation

In some embodiments, the polarization transfer magnetic perturbation is performed in a magnetic shield (e.g., a mu shield, or the like) to achieve a homogenous, low magnetic field. The magnetic shield enables performance of polarization transfer to ¹³C nuclear spins at microtesla (μT) magnetic fields, below the earth's magnetic field. The low magnetic field can be at least about 0 mG, 0.1 mG, 0.2 mG, 0.3 mG, 0.4 mG, 0.5 mG, 0.6 mG, 0.7 mG, 0.8 mG, 0.9 mG, 1 mG, 2 mG, 3 mG, 4 mG, 5 mG, 6 mG, 7 mG, 8 mG, 9 mG, 10 mG, 20 mG, 30 mG, 40 mG, 50 mG, 60 mG, 70 mG, 80 mG, 90 mG, 100 mG, or more, at most about 100 mG, 90 mG, 80 mG, 70 mG, 60 mG, 50 mG, 40 mG, 30 mG, 20 mG, 10 mG, 9 mG, 8 mG, 7 mG, 6 mG, 5 mG, 4 mG, 3 mG, 2 mG, 1 mG, 0.9 mG, 0.8 mG, 0.7 mG, 0.6 mG, 0.5 mG, 0.4 mG, 0.3 mG, 0.2 mG, 0.1 mG, or less, or within a range defined by any two of the preceding values.

At such fields, the polarization is transferred by utilizing level avoided crossings (LAC) between the proton spins and other spin species of interest, including ²H, ¹³C, ¹⁵N, ¹⁹F, and ³¹P. In some embodiments, the magnetic field can be tuned to a specific magnetic field strength for the LAC, for example as performed in SABRE-SHEATH experiments. In various embodiments, to enable robust polarization transfer in larger-volume samples, the magnetic field strength can be temporally modulated. For example, the magnetic field strength can be swept through the LAC conditions. Alternatively or additionally, the sample can be physically moved inside the magnetic field. Such modulation can relax constraints on magnetic field homogeneity and on magnetic field offsets. Thus, robust polarization transfer can be performed at larger volumes and with greater efficiency. Furthermore, relaxing the constraints on magnetic field homogeneity and on magnetic field offsets can permit using of less complex, precise, or expensive polarization systems.

A lower bound of the magnetic field modulation can at least about −10 μT, −9 μT, −8 μT, −7 μT, −6 μT, −5 μT, −4 μT, −3 μT, −2 μT, −1.9 μT, −1.8 μT, −1.7 μT, −1.6 μT, −1.5 μT, −1.4 μT, −1.3 μT, −1.2 μT, −1.1 μT, −1 μT, −0.9 μT, −0.8 μT, −0.7 μT, −0.6 μT, −0.5 μT, −0.4 μT, −0.3 μT, −0.2 μT, −0.1 μT, or more, at most about −0.1 μT, −0.2 μT, −0.3 μT, −0.4 μT, −0.5 μT, −0.6 μT, −0.7 μT, −0.8 μT, −0.9 μT, −1 μT, −1.1 μT, −1.2 μT, −1.3 μT, −1.4 μT, −1.5 μT, −1.6 μT, −1.7 μT, −1.8 μT, −1.9 μT, −2 μT, −3 μT, −4 μT, −5 μT, −6 μT, −7 μT, −8 μT, −9 μT, −10 μT, or less, or within a range defined by any two of the preceding values. An upper bound of the modulation can be at least about 0.1 μT, 0.2 μT, 0.3 μT, 0.4 μT, 0.5 μT, 0.6 μT, 0.7 μT, 0.8 μT, 0.9 μT, 1 T, 1.1 μT, 1.2 μT, 1.3 μT, 1.4 μT, 1.5 μT, 1.6 μT, 1.7 μT, 1.8 μT, 1.9 μT, 2 μT, 3 μT, 4 μT, 5 T, 6 μT, 7 μT, 8 μT, 9 μT, 10 μT, or more, at most about 10 μT, 9 μT, 8 μT, 7 μT, 6 μT, 5 μT, 4 μT, 3 μT, 2 μT, 1.9 μT, 1.8 μT, 1.7 μT, 1.6 μT, 1.5 μT, 1.4 μT, 1.3 μT, 1.2 μT, 1.1 μT, 1 μT, 0.9 μT, 0.8 μT, 0.7 μT, 0.6 μT, 0.5 μT, 0.4 μT, 0.3 μT, 0.2 μT, 0.1 μT, or less, or within a range defined by any two of the preceding values.

The magnetic field can have such an amplitude over a volume of at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 200 ml, 300 ml, 400 ml, 500 ml, 600 ml, 700 ml, 800 ml, 900 ml, 1,000 ml, 2,000 ml, or more, at most about 2,000 ml, 1,000 ml, 900 ml, 800 ml, 700 ml, 600 ml, 500 ml, 400 ml, 300 ml, 200 ml, 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less, or a volume that is within a range defined by any two of the preceding values. The modulation can be performed over a duration. The duration can be at least about 100 milliseconds (ms), 200 ms, 300 ms, 400 ms, 500 ms, 600 ms, 700 ms, 800 ms, 900 ms, 1 second (s), 2 s, 3 s, 4 s, 5 s, 6 s, 7 s, 8 s, 9 s, 10 s, 20 s, 30 s, or more, at most about 30 s, 20 s, 10 s, 9 s, 8 s, 7 s, 6 s, 5 s, 4 s, 3 s, 2 s, 1 s, 900 ms, 800 ms, 700 ms, 600 ms, 500 ms, 400 ms, 300 ms, 200 ms, 100 ms, or less, or within a range defined by any two of the preceding values.

Accordingly, the rate of change of the amplitude of the magnetic field can be at least about 0.01 μT per second, 0.015 μT per second, 0.02 μT per second, 0.025 μT per second, 0.03 μT per second, 0.035 μT per second, 0.04 μT per second, 0.045 μT per second, 0.05 μT per second, 0.055 μT per second, 0.06 μT per second, 0.065 μT per second, 0.07 μT per second, 0.075 μT per second, 0.08 μT per second, 0.085 μT per second, 0.09 μT per second, 0.095 μT per second, 0.1 μT per second, 0.15 μT per second, 0.2 μT per second, 0.25 μT per second, 0.3 μT per second, 0.35 μT per second, 0.4 μT per second, 0.45 μT per second, 0.5 μT per second, 0.55 μT per second, 0.6 μT per second, 0.65 μT per second, 0.7 μT per second, 0.75 μT per second, 0.8 μT per second, 0.85 μT per second, 0.9 μT per second, 0.95 μT per second, 1 μT per second, or more, at most about 1 μT per second, 0.95 μT per second, 0.9 μT per second, 0.85 μT per second, 0.8 μT per second, 0.75 μT per second, 0.7 μT per second, 0.65 μT per second, 0.6 μT per second, 0.55 μT per second, 0.5 μT per second, 0.45 μT per second, 0.4 μT per second, 0.35 μT per second, 0.3 μT per second, 0.25 μT per second, 0.2 μT per second, 0.15 μT per second, 0.1 μT per second, 0.095 μT per second, 0.09 μT per second, 0.08 μT per second, 0.075 μT per second, 0.07 μT per second, 0.065 μT per second, 0.06 μT per second, 0.055 μT per second, 0.05 μT per second, 0.045 μT per second, 0.04 μT per second, 0.035 μT per second, 0.03 μT per second, 0.025 μT per second, 0.02 μT per second, 0.015 μT per second, 0.01 μT per second, or less, or within a range defined by any two of the preceding values. The upper bound on the rate of change of the amplitude of the magnetic field may be determined by the capabilities of the equipment used to perform the sweep.

In some embodiments, when the magnetic field is within the upper and lower bounds, disclosed above, the spatial deviation of the magnetic field over the volume during modulation is less than about half (or a quarter, or an eighth, or a tenth) of the amplitude of the magnetic field. For example, when the magnetic field strength is less than 2 μT (or greater than −2 μT) then the spatial deviation of the magnetic field over the volume during modulation can be less than 1 μT. As an additional example, when the magnetic field strength is less than 10 μT (or greater than −10 μT) then the spatial deviation of the magnetic field over the volume during modulation can be less than 5 μT. The spatial deviation can be measured for example by taking at least about 10, 20, 30, 40, 50, 60, 70, 80, 90, 100, 200, 300, 400, 500, or more spatially randomly sampled or spatially equally distributed measurements of the magnetic field within the volume and calculating the standard deviation of the sampled magnetic field measurements.

Such homogeneity can be achieved for example in a large homogeneous magnetic shield by having a large piercing solenoid through the magnetic shield or by using large Helmholtz coils with a large homogeneous region for producing the magnetic field amplitude modulation. In some embodiments the modulation is a sweep of the magnetic field. In some embodiments, the magnetic field amplitude modulation includes a diabatic jump, monotonous amplitude variation or combinations thereof.

In some embodiments, following the polarization transfer step, a non-hydrogen nuclear spin of the biorelevant imaging agent (such as a ¹³C or ¹⁵N of the biorelevant imaging agent) has nuclear spin polarization of at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1% or less, or a polarization that is within a range defined by any two of the preceding values. For example, in some embodiments, following the polarization transfer step, a non-hydrogen nuclear spin of the biorelevant imaging agent has nuclear spin polarization between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%.

In some embodiments this polarization is achieved for a solution volume of at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, 200 ml, 300 ml, 400 ml, 500 ml, or more, at most about 500 ml, 400 ml, 300 ml, 200 ml, 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less, or a volume that is within a range defined by any two of the preceding values.

In some embodiments, following polarization transfer a portion of the population difference in parahydrogenated proton spin states has been transferred to polarization of the target (e.g., ¹³C or ¹⁵N) nuclear spin of the biorelevant imaging agent. This portion can be at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6% 5%, 4%, 3%, 2%, 1% or less, or within a range defined by any two of the preceding values. For example, in some embodiments, this portion is between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%.

In some embodiments, the magnetic field modulation includes a diabatic jump of the magnetic field. The diabatic jump can be performed to a magnetic field where a level avoided crossing including the proton spins and a non-proton spin occur. Given the J-couplings between the nuclear spins in the system, this value can be calculated analytically or identified by plotting the energy levels of the Hamiltonian for different magnetic fields and identifying the LAC. In some embodiments, the duration where the magnetic field amplitude is at the LAC condition is at most about 5 seconds, 4 seconds, 3 seconds, 2 seconds, 1 second, 0.9 seconds, 0.8 seconds, 0.7 seconds, 0.6 seconds, 0.5 seconds, 0.4 seconds, 0.3 seconds, 0.2 seconds, 0.1 seconds, or less, at least about 0.1 seconds, 0.2 seconds, 0.3 seconds, 0.4 seconds, 0.5 seconds, 0.6 seconds, 0.7 seconds, 0.8 seconds, 0.9 seconds, 1 seconds, 2 seconds, 3 seconds, 4 seconds, 5 seconds, or more or within a range defined by any two of the preceding values.

In some embodiments, modulation of the amplitude of the magnetic field includes varying the magnetic field amplitude monotonically (or monotonically over each of a limited number of interval—such as one to ten increasing interval and/or one to ten decreasing intervals). In some embodiments, the modulation of the amplitude of the magnetic field comprises linearly varying the amplitude of the magnetic field. The initial magnetic field amplitude of the sweep, the end magnetic field amplitude and the total duration of the sweep can be optimized for the target molecule. In some embodiments the magnetic field amplitude during the sweep is within a lower bound and an upper bound. The lower bound can be at least about −2 μT, −1.9 μT, −1.8 μT, −1.7 μT, −1.6 μT, −1.5 μT, −1.4 μT, −1.3 μT, −1.2 μT, −1.1 μT, −1 μT, −0.9 μT, −0.8 μT, −0.7 μT, −0.6 μT, −0.5 μT, −0.4 μT, −0.3 μT, −0.2 μT, −0.1 μT, or more, at most about −0.1 μT, −0.2 μT, −0.3 μT, −0.4 μT, −0.5 μT, −0.6 μT, −0.7 μT, −0.8 μT, −0.9 μT, −1 μT, −1.1 μT, −1.2 μT, −1.3 μT, −1.4 μT, −1.5 μT, −1.6 μT, −1.7 μT, −1.8 μT, −1.9 μT, −2 μT, or less, or within a range defined by any two of the preceding values. The upper bound can be at least about 0.1 μT, 0.2 μT, 0.3 μT, 0.4 μT, 0.5 μT, 0.6 μT, 0.7 μT, 0.8 μT, 0.9 μT, 1 μT, 1.1 μT, 1.2 μT, 1.3 μT, 1.4 μT, 1.5 μT, 1.6 μT, 1.7 μT, 1.8 μT, 1.9 μT, 2 μT, or more, at most about 2 T, 1.9 μT, 1.8 μT, 1.7 μT, 1.6 μT, 1.5 μT, 1.4 μT, 1.3 μT, 1.2 μT, 1. T, 1 μT, 0.9 μT, 0.8 μT, 0.7 μT, 0.6 μT, 0.5 μT, 0.4 μT, 0.3 μT, 0.2 μT, 0.1 μT, or less, or within a range defined by any two of the preceding values. In some embodiments the duration of modulation can be at least about 100 ms, 200 ms, 300 ms, 400 ms, 500 ms, 600 ms, 700 ms, 800 ms, 900 ms, 1 s, 2 s, 3 s, 4 s, 5 s, 6 s, 7 s, 8 s, 9 s, 10 s, 20 s, 30 s, or more, at most about 30 s, 20 s, 10 s, 9 s, 8 s, 7 s, 6 s, 5 s, 4 s, 3 s, 2 s, 1 s, 900 ms, 800 ms, 700 ms, 600 ms, 500 ms, 400 ms, 300 ms, 200 ms, 100 ms, or less, or within a range defined by any two of the preceding values. In some embodiments, the rate of amplitude change is varied along the amplitude profile. In some embodiments, a constant-adiabaticity sweep is calculated by choosing a certain subset of level avoided crossings of the spin system. In some embodiments, the magnetic amplitude modulation includes a combination of diabatic jumps, monotonous amplitude modulation and rate of change sign reversals. In some embodiments, the precursor may be chosen or designed such that following the hydrogenation and other potential chemical reactions, one of the products is a biorelevant imaging agent usable in hyperpolarized NMR or MRI applications.

### Hydrolysis, Purification and Separation

The present disclosure presents methods and systems for producing a composition (e.g., clinical dose composition) which comprises a hyperpolarized biorelevant imaging agent (or a pharmaceutically acceptable salt thereof) in a solvent. In some embodiments, the biorelevant imaging agent is produced through additional chemical reactions and/or processing steps following hydrogenation and polarization transfer, according to the present disclosure. Such additional chemical reactions and/or processing steps may include, but are not limited to: (i) catalyst filtration and scavenging (e.g., filtration scavenging rhodium atoms and/or iridium atoms); (ii) cleaving the sidearm of the biorelevant imaging agent precursor molecule (e.g., cleavage of the compound of Formula IIa or Formula IIb, as described herein) to form the biorelevant imaging agent and a sidearm (e.g., a compound of Formula IIIa or Formula IIIb described herein), e.g., by hydrolysis with an aqueous sodium hydroxide solution; (iii) washing the solution with an organic solvent and separating any resulting aqueous mixture phase from an organic mixture phase; (iv) evaporative extraction of volatile organics from the aqueous mixture (e.g., using nitrogen gas bubbling); and (v) additional filtration/purification/concentration/finishing steps known in the art.

The volume of the solution which includes the biorelevant imaging agent, (e.g., following cleavage) and/or the concentration of the biorelevant imaging agent produced can depend on the volume of the solution used for polarization transfer and concentration of the precursor in that solution. Exemplary ranges of solution volumes and precursor concentrations are described herein. As further specific examples, at least about 1 ml, 2 ml, 3 ml, 4 ml, 5 ml, 6 ml, 7 ml, 8 ml, 9 ml, 10 ml, 20 ml, 30 ml, 40 ml, 50 ml, 60 ml, 70 ml, 80 ml, 90 ml, 100 ml, or more of solution, at most about 100 ml, 90 ml, 80 ml, 70 ml, 60 ml, 50 ml, 40 ml, 30 ml, 20 ml, 10 ml, 9 ml, 8 ml, 7 ml, 6 ml, 5 ml, 4 ml, 3 ml, 2 ml, 1 ml, or less of solution, or an amount of solution that is within a range defined by any two of the preceding values can be produced. In some embodiments, the solution can include at least about 10 mM, 20 mM, 30 mM, 40 mM, 50 mM, 60 mM, 70 mM, 80 mM, 90 mM, 100 mM, 200 mM, 300 mM, 400 mM, 500 mM, or more of the biorelevant imaging agent, at most about 500 mM, 400 mM, 300 mM, 200 mM, 100 mM, 90 mM, 80 mM, 70 mM, 60 mM, 50 mM, 40 mM, 30 mM, 20 mM, 10 mM, or less of the biorelevant imaging agent, or an amount of the biorelevant imaging agent this is within a range defined by any two of the preceding values.

In some embodiments, the present disclosure describes a multi-step liquid-liquid separation and purification process for producing doses (e.g., clinical doses) of a dosage composition comprising the biorelevant imaging agent (e.g., hyperpolarized biorelevant imaging agent or a pharmaceutically acceptable salt thereof).

In some embodiments (i.e., for PHIP-SAH procedures), the polarization step is followed by the sidearm being cleaved (e.g., via hydrolysis with an aqueous mixture) from the target molecule precursor (e.g., biorelevant imaging agent precursor) to produce a target molecule (e.g., biorelevant imaging agent) and an unbound sidearm (such as a compound of Formula IIIa, as described herein). In some embodiments, the polarization step is followed by the sidearm being cleaved by mixing the solution (which includes the first organic solvent and the polarized product, e.g., hyperpolarized biorelevant imaging agent or a pharmaceutically acceptable salt thereof) with a hydrolyzing agent, such as a base (e.g., sodium hydroxide) in an aqueous solution. In some embodiments, the first organic solvent and the aqueous mixture (e.g., water) produce a biphasic solution. In some embodiments, the first organic solvent and aqueous mixture (e.g., water) produce a biphasic solution, wherein a portion of the organic solvent is retained in the aqueous mixture. In some embodiments, the first organic solvent and aqueous mixture (e.g., water) produce a partial mixture.

### Clinically Relevant Purities

Consistent with disclosed embodiments, steps the methods and systems described herein can separate the hyperpolarized biorelevant imaging agent from other substances in the original solution (e.g., catalysts, the original solvent(s), reaction products, or the like). For example, most of the hydrogenation catalyst present in the original solution can be removed from the dosage composition. In some embodiments, the dosage composition can retain at most about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less of the hydrogenation catalyst, at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, or more of the hydrogenation catalyst, or an amount of the hydrogenation catalyst that is within a range defined by any two of the preceding values. Similarly, the dosage composition can retain at most about 1%, 0.9%, 0.8%, 0.7%, 0.6%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, or less of the cleavage byproducts (e.g., the sidearm or other residues of the cleavage), at least about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 10%, or more of the cleavage byproducts, or an amount of the cleavage byproducts that is within a range defined by any two of the preceding values.

In some embodiments, the methods and systems described herein produce dosage compositions in which the concentration of the hyperpolarized biorelevant imaging is at least about 100 mM, 150 mM, 200 mM, 250 mM, 300 mM, 350 mM, 400 mM, 450 mM, 500 mM, or more, at most about 500 mM, 450 mM, 400 mM, 350 mM, 300 mM, 250 mM, 200 mM, 150 mM, 100 mM, or less, or within a range defined by any two of the preceding values.

In some embodiments, the methods and systems described herein produce dosage compositions in which the polarization of the hyperpolarized biorelevant imaging is at least about 1%, 2%, 3%, 4%, 5%, 6%, 7%, 8%, 9%, 10%, 15%, 20%, 25%, 30%, 40%, 45%, 50%, more, at most about 50%, 45%, 40%, 35%, 30%, 25%, 20%, 15%, 10%, 9%, 8%, 7%, 6%, 5%, 4%, 3%, 2%, 1%, or less, or polarization that is within a range defined by any two of the preceding values. For example, in some embodiments, the methods and systems described herein produce dosage compositions in which the polarization of the hyperpolarized biorelevant imaging is between 10% and 15%, between 10% and 20%, between 10% and 25%, between 10% and 30%, between 10% and 35%, between 10% and 40%, between 10% and 45%, between 10% and 50%, between 15% and 20%, between 15% and 25%, between 15% and 30%, between 15% and 35%, between 15% and 40%, between 15% and 45%, between 15% and 50%, between 20% and 25%, between 20% and 30%, between 20% and 35%, between 20% and 40%, between 20% and 45%, between 20% and 50%, between 25% and 30%, between 25% and 35%, between 25% and 40%, between 25% and 45%, between 25% and 50%, between 30% and 35%, between 30% and 40%, between 30% and 45%, between 30% and 50%, between 35% and 40%, between 35% and 45%, between 35% and 50%, between 40% and 45%, between 40% and 50%, or between 45% and 50%.

In some embodiments, the methods and systems described herein produce dosage compositions in which the concentration of catalysts, the precursor, or the cleavage byproducts may each be at most about 1 μM, 900 nanomolar (nM), 800 nM, 700 nM, 600 nM, 500 nM, 400 nM, 300 nM, 200 nM, 100 nM, 90 nM, 80 nM, 70 nM, 60 nM, 50 nM, 40 nM, 30 nM, 20 nM, 10 nM, 9 nM, 8 nM, 7 nM, 6 nM, 5 nM, 4 nM, 3 nM, 2 nM, 1 nM, or less, at least about 1 nM, 2 nM, 3 nM, 4 nM, 5 nM, 6 nM, 7 nM, 8 nM, 9 nM, 10 nM, 20 nM, 30 nM, 40 nM, 50 nM, 60 nM, 70 nM, 80 nM, 90 nM, 100 nM, 200 nM, 300 nM, 400 nM, 500 nM, 600 nM, 700 nM, 800 nM, 900 nM, 1 μM, or more, or within a range defined by any two of the preceding values. the methods and systems described herein produce dosage compositions in which the purity of the hyperpolarized biorelevant imaging is at least about 90% 91%, 92%, 93%, 94%, 95%, 96%, 97%, 98%, 99%, or more, at most about 99%, 98%, 97%, 96%, 95%, 94%, 93%, 92%, 91%, 90%, or less, or within a range defined by any two of the preceding values. In some embodiments, at least a fraction of the hyperpolarized compounds is separated from the cleaved sidearms, or other reaction byproducts, if such exist.

### Transportation

Consistent with disclosed embodiments, polarization transfer and use of the biorelevant imaging agent can occur at different locations. In some embodiments, the dosage composition is transported to another location. In some embodiments, the dosage composition is transported to another location. The disclosed embodiments are not necessarily limited to any particular transport distance or duration. Instead, a maximum distance or duration can be determined based on the target molecule, the original degree or polarization, the required final degree of polarization, and the transport conditions. In some embodiments, the dosage composition is transported at least one meter in a suitable transportation device.

Consistent with disclosed embodiments, a transportation device can be configured to transport samples of the precursor or biorelevant imaging agent. The transportation device can be arranged and configured for transporting one or more samples (e.g., one or more dosage compositions) simultaneously. The transportation device can include a transport chamber configured to receive the one or more samples. The transportation device can be configured to maintain the transport chamber within a predetermined temperature range and a predetermined magnetic field strength. The transportation device can be configured to maintain the one or more samples in a magnetic field of at least about 10 G, 20 G, 30 G, 40 G, 50 G, 60 G, 70 G, 80 G, 90 G, 100 G, 200 G, 300 G, 400 G, 500 G, 600 G, 700 G, 800 G, 900 G, 1,000 G, or more, at most about 1,000 G, 900 G, 800 G, 700 G, 600 G, 500 G, 400 G, 300 G, 200 G, 100 G, 90 G, 80 G, 70 G, 60 G, 50 G, 40 G, 30 G, 20 G, 10 G, or less, or within a magnetic field that is within a range defined by any two of the previous values.

A permanent magnet or an electromagnet included in the transportation device can provide the magnetic field. In some embodiments, the permanent magnet or electromagnet is shielded to reduce the strength of the magnetic field outside the transportation device. The transportation device can also include a cooling system. The cooling system can be configured to maintain samples at a predetermined temperate or within a predetermined range of temperatures during transport. For example, the cooling system can be configured to maintain the samples at a temperature below 270 K, below 80 K, or below 4 K. In some embodiments, the transportation device is configured to maintain the samples at approximately the temperature of liquid nitrogen. The transportation device can include insulation between the cooling system and the exterior of the transportation device, to minimize heat exchange with the external environment. In some embodiments, the cooling system is configured to maintain the temperature of the samples using a cold gas flow. In some embodiments, the cooling system is configured to maintain the temperature of the samples using a liquid coolant. In some embodiments, the transportation device includes a Dewar to provide cooling of the samples. In order to distribute the hyperpolarized samples also across large distances, the container can be transported by standard transportation vehicles, such as planes, trains, trucks, cars and ships.

In some embodiments, the dosage composition containing the hyperpolarized biorelevant imaging agent is transported in the transportation device. In some embodiments the relaxation time of the hyperpolarized biorelevant imaging agent in the transportation device at least about 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, 1 hour, 2 hours, 3 hours, 4 hours, 5 hours, 6 hours, 7 hours, 8 hours, 9 hours, 10 hours, or more, at most about 10 hours, 9 hours, 8 hours, 7 hours, 6 hours, 5 hours, 4 hours, 3 hours, 2 hours, 1 hour, 50 minutes, 40 minutes, 30 minutes, 20 minutes, 10 minutes, 9 minutes, 8 minutes, 7 minutes, 6 minutes, 5 minutes, 4 minutes, 3 minutes, 2 minutes, 1 minute, or less, or a relaxation time that is within a range defined by any two of the preceding values.

### Generation of Polarized Biorelevant Imaging Agents

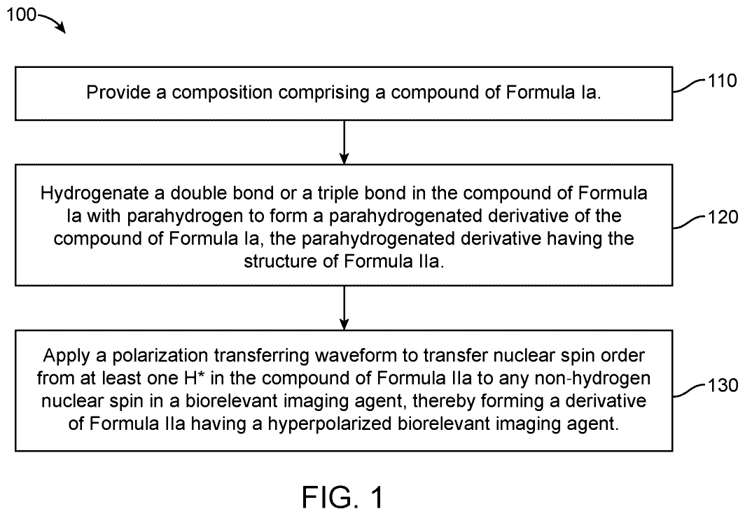

FIG. 1 depicts a first exemplary process 100 for generating polarized biorelevant imaging agents, in accordance with various embodiments. In some embodiments, the first process 100 comprises providing a composition comprising a compound of Formula Ia at step 110. In some embodiments, the compound of Formula Ia comprises: a Z group comprising: (i) a carbon-carbon double bond (—C═C—) which is fully substituted to ²H (deuterium, also referred to as D) (i.e., —CD=CD-) or (ii) a carbon-carbon triple bond (—C—C—), as described herein; an R₁ group comprising a PHIP transfer moiety descried herein; an R₂ group comprising an optionally substituted hydrocarbon, alkoxy group, primary amine, secondary amine, or tertiary amine, or a solubilizing moiety, as described herein; and an R₃ group comprising a biorelevant imaging agent, as described herein.

In some embodiments, at step 120, the double bond or the triple bond in the compound of Formula Ia is hydrogenated with parahydrogen to form a parahydrogenated derivative of the compound of Formula Ia, wherein the parahydrogenated derivative is a compound having the structure of Formula IIa. In some embodiments, the compound of Formula IIa comprises: a Z′ which is: (i) a parahydrogenated carbon-carbon single bond (—CH*—CH*—) which is fully substituted to include ²H (deuterium, also referred to as D) (i.e., —CDH*—CDH*—), or (ii) a parahydrogenated carbon-carbon double bond (—CH*═CH*—), wherein H* is a hydrogen having a spin order derived from parahydrogen; an R₁ group comprising a PHIP transfer moiety, as described herein; an R₂ group comprising an optionally substituted hydrocarbon, alkoxy group, primary amine, secondary amine, or tertiary amine, or a solubilizing moiety, as described herein; and an R₃ group comprising a biorelevant imaging agent, as described herein. In some embodiments, the compound of Formula Ia is hydrogenated with parahydrogen using a hydrogenation process described herein.

In some embodiments, at step 130, a polarization transferring waveform is applied to transfer nuclear spin order from at least one H* in the sidearm of the compound of Formula IIa to any non-hydrogen nuclear spin in the biorelevant imaging agent of the compound of Formula IIa, as described herein, thereby forming a derivative of the compound of Formula IIa having a hyperpolarized biorelevant imaging agent. In some embodiments, the nuclear spin order is transferred using any polarization transfer process described herein.

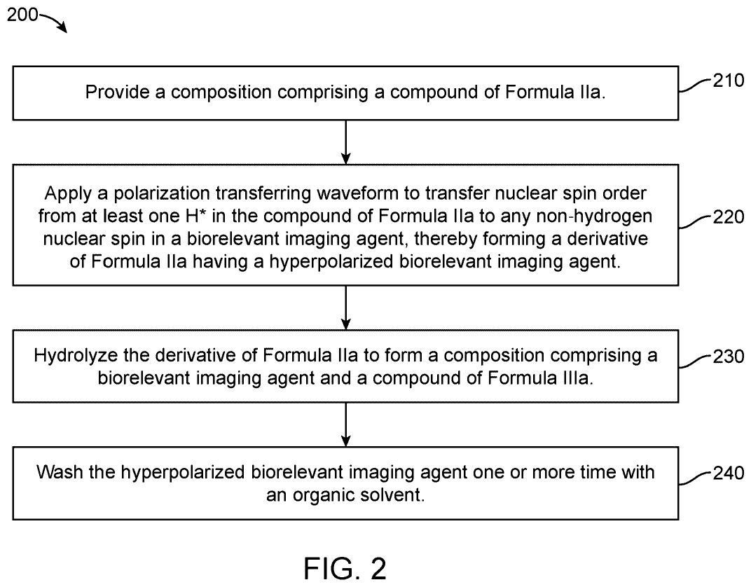

FIG. 2 depicts a second exemplary process 200 for generating polarized biorelevant imaging agents, in accordance with various embodiments of the present disclosure. In some embodiments, the second process comprises providing a composition comprising a compound of Formula IIa at step 210. In some embodiments, Formula IIa comprises a Z′ group which is: (i) a parahydrogenated carbon-carbon single bond (—CH*—CH*—) which is fully substituted to include ²H (deuterium, also referred to as D) (i.e., —CDH*—CDH*—), or (ii) a parahydrogenated carbon-carbon double bond (—CH*═CH*—), as described herein, wherein H* is a hydrogen having a spin order derived from parahydrogen; an R₁ group comprising a PHIP transfer moiety, as described herein; an R₂ group comprising an optionally substituted hydrocarbon, alkoxy group, primary amine, secondary amine, or tertiary amine, or a solubilizing moiety, as described herein; and an R₃ group comprising a biorelevant imaging agent, as described herein.

In some embodiments, at step 220, a polarization transferring waveform is applied to the transfer nuclear spin order from at least one H* in the sidearm of the compound of Formula IIa to any non-hydrogen nuclear spin in the biorelevant imaging agent of the compound of Formula IIa, as descried herein, thereby forming a derivative of the compound of Formula IIa having a hyperpolarized biorelevant imaging agent.

In some embodiments, at step 230, the derivative compound of Formula IIa is hydrolyzed to form a composition comprising a hyperpolarized biorelevant imaging agent and a separate sidearm compound of Formula IIIa. In some embodiments, the compound of Formula IIIa comprises a Z″ which is: (i) a parahydrogenated carbon-carbon single bond (—CH*—CH*—) which is fully substituted to include ²H (deuterium, also referred to as D) (i.e., —CDH*—CDH*), or (ii) a parahydrogenated carbon-carbon double bond (—CH*═CH*—), as described herein; an R₁′ group comprising a parahydrogen induced polarization (PHIP) transfer moiety, as described herein; and an R₂ group comprising an optionally substituted hydrocarbon, alkoxy group, primary amine, secondary amine, or tertiary amine, or a solubilizing moiety, as described herein.

In some embodiments, at step 240, the hyperpolarized biorelevant imaging agent is washed one or more times with an organic solvent. In some embodiments, the non-hydrogen nuclear spin in the biorelevant imaging agent has a non-hydrogen spin polarization after the washing step of at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1%, or less, or a non-hydrogen spin polarization that is within a range defined by any two of the preceding values.

In some embodiments, the first or second process comprises one or more additional steps or operations. In some embodiments, the first or second process omits one or more steps or operations. In some embodiments, one or more steps or operations of the first or second process are combined. In some embodiments, all steps or operations of the first or second process are combined to yield a complete process for generating a hyperpolarized imaging agent from a precursor having the structure of Formula Ia.

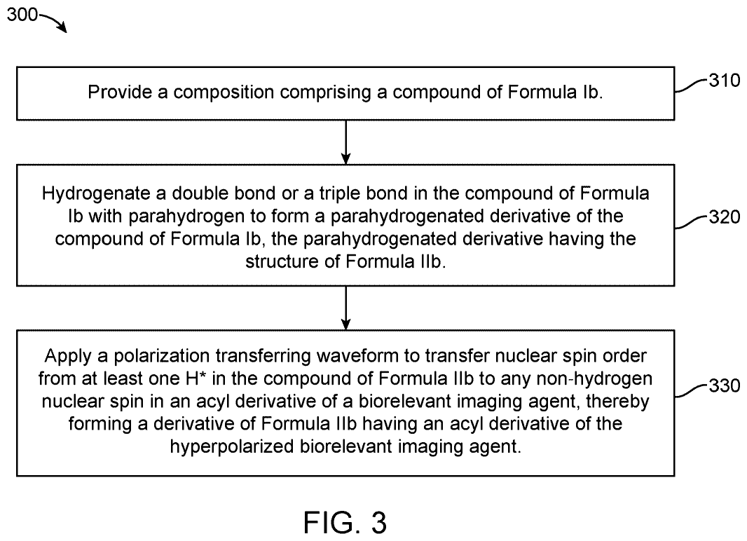

FIG. 3 depicts a third exemplary process 300 for generating polarized biorelevant imaging agents, in accordance with various embodiments. In the example shown, the process comprises providing a composition comprising a compound of Formula Ib at step 310. In some embodiments, the compound of Formula Ib comprises a Z group comprising an ethynyl (—C—C—) group, a fully deuterated prop-2-ynyl (—CD₂-C≡C—) group, a fully deuterated ethenyl (—CD=CD-) group, an a fully deuterated prop-2-enyl (—CD₂-CD=CD-) group, or a fully deuterated but-3-enyl (—CD₂-CD₂-CD=CD-) group, as described herein, an R₂ group comprising an optionally substituted hydrocarbon group, alkyl group, cyclic alkyl group, aryl group, carboxyl group, keto group, or a solubilizing moiety, as described herein, and an R₃ group comprising an acyl derivative of a biorelevant imaging agent, as described herein.

In some embodiments, at step 320, the double bond or the triple bond in the compound of Formula Ib is hydrogenated with parahydrogen to form a parahydrogenated derivative of the compound of Formula Ib, wherein the parahydrogenated derivative is a compound having the structure of Formula IIb. In some embodiments, the compound of Formula IIb comprises a Z′ group comprising a parahydrogenated ethenyl (—CH*═CH*—) group, a fully deuterated parahydrogenated prop-2-enyl (—CD₂-CH*═CH*—) group, a fully deuterated parahydrogenated ethanyl (—CDH*—CDH*—) group, a fully deuterated parahydrogenated propanyl (—CD₂-CDH*—CDH*—) group, or a fully deuterated parahydrogenated butanyl (—CD₂-CD₂-CH*═CH*—) group, wherein H* is a hydrogen having a spin order derived from parahydrogen, as described herein, an R₂ group comprising an optionally substituted hydrocarbon group, alkyl group, cyclic alkyl group, aryl group, carboxyl group, keto group, or alkoxy group or a solubilizing moiety, as described herein, and an R₃ group comprising an acyl derivative of a biorelevant imaging agent, as described herein. In some embodiments, the compound of Formula Ib is hydrogenated with parahydrogen using a hydrogenation process described herein.

In some embodiments, at step 330, a polarization transferring waveform is applied to transfer nuclear spin order from at least one H* in the sidearm of the compound of Formula IIb to any non-hydrogen nuclear spin in the acyl derivative of the biorelevant imaging agent of the compound of Formula IIb, as described herein, thereby forming a derivative of the compound of Formula IIb having a hyperpolarized acyl derivative of the biorelevant imaging agent. In some embodiments, the nuclear spin order is transferred using any polarization transfer process described herein.

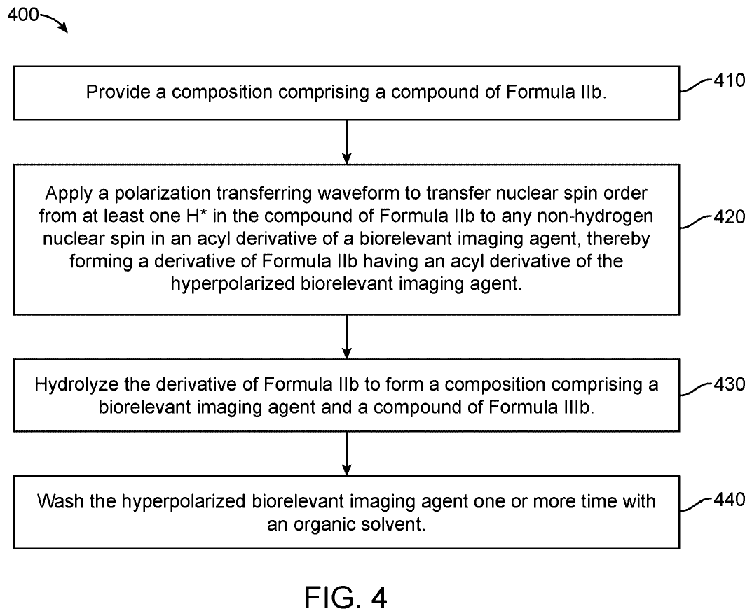

FIG. 4 depicts a fourth exemplary process 400 for generating polarized biorelevant imaging agents, in accordance with various embodiments of the present disclosure. In the example shown, the process comprises providing a composition comprising a compound of Formula IIb at step 410. In some embodiments, Formula IIb comprises a Z′ group comprising a parahydrogenated ethenyl (—CH*═CH*—) group, a fully deuterated parahydrogenated prop-2-enyl (—CD₂-CH*═CH*—) group, a fully deuterated parahydrogenated ethanyl (—CDH*—CDH*—) group, a fully deuterated parahydrogenated propanyl (—CD₂-CDH*—CDH*—) group, or a fully deuterated butanyl (—CD₂-CD₂-CH*═CH*—) group, as described herein, wherein H* is a hydrogen having a spin order derived from parahydrogen, an R₂ group comprising an optionally substituted hydrocarbon group, alkyl group, cyclic alkyl group, aryl group, carboxyl group, keto group, or alkoxy group or a solubilizing moiety, as described herein, and an R₃ group comprising an acyl derivative of a biorelevant imaging agent, as described herein.

In some embodiments, at step 420, a polarization transferring waveform is applied to the transfer nuclear spin order from at least one H* in the sidearm of the compound of Formula IIb to any non-hydrogen nuclear spin in the acyl derivative of the biorelevant imaging agent of the compound of Formula IIb, as descried herein, thereby forming a derivative of the compound of Formula IIb having a hyperpolarized acyl derivative of the biorelevant imaging agent.

In some embodiments, at step 430, the derivative compound of Formula IIb is hydrolyzed to form a composition comprising a hyperpolarized biorelevant imaging agent and a separate sidearm compound of Formula IIIb. In some embodiments, the compound of Formula IIIb comprises a Z″ group comprising a parahydrogenated ethenyl (—CH*═CH*—) group, a fully deuterated parahydrogenated prop-2-enyl (—CD₂-CH*═CH*—) group, a fully deuterated parahydrogenated ethanyl (—CDH*—CDH*—) group, a fully deuterated parahydrogenated propanyl (—CD₂-CDH*—CDH*—) group, or a fully deuterated parahydrogenated butanyl (—CD₂-CD₂-CH*═CH*—) group, as described herein, and an R₂ group comprising an optionally substituted hydrocarbon group, alkyl group, cyclic alkyl group, aryl group, carboxyl group, keto group, or alkoxy group or a solubilizing moiety, as described herein.

In some embodiments, at step 440, the hyperpolarized biorelevant imaging agent is washed one or more times with an organic solvent. In some embodiments, the non-hydrogen nuclear spin in the biorelevant imaging agent has a non-hydrogen spin polarization after the washing step of at least about 1%, 2%, 3%, 4%, 5%, 6%, 7%, 8%, 9%, 10%, 15%, 20%, 25%, 30%, 35%, 40%, 45%, 50%, or more, at most about 50%, 45%, 40%, 35%, 30%, 25%, 20%, 15%, 10%, 9%, 8%, 7%, 6%, 5%, 4%, 3%, 2%, 1%, or less, or a non-hydrogen spin polarization that is within a range defined by any two of the preceding values.

In some embodiments, the third or fourth process comprises one or more additional steps or operations. In some embodiments, the third or fourth process omits one or more steps or operations. In some embodiments, one or more steps or operations of the third or fourth process are combined. In some embodiments, all steps or operations of the third or fourth process are combined to yield a complete process for generating a hyperpolarized imaging agent from a precursor having the structure of Formula Ib.

### EXAMPLES

### Example 1: Polarization Transfer in Non-Deuterated, Partially Deuterated, and Fully Deuterated Pyruvate Derivatives

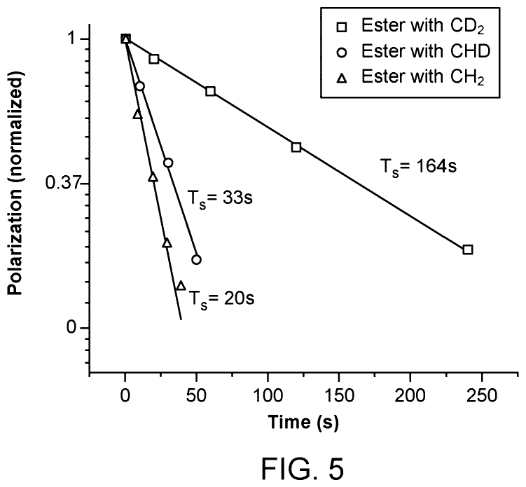

To assess the impact of deuteration level on a PHIP transfer moiety in pyruvate, the following ester sidearm derivatives of pyruvate were prepared: (1) tert-butyl 4-((2-oxopropanoyl)oxy)but-2-ynoate (CH₂ ester), (2) tert-butyl 4-((2-oxopropanoyl)oxy)but-2-ynoate-D1 (CDH ester), and (3) tert-butyl 4-((2-oxopropanoyl)oxy)but-2-ynoate-D2 (CD₂ ester). Compounds (1), (2), and (3) were prepared with natural abundance ¹³C (about 1.1%). Compounds (1), (2), and (3) were reacted with parahydrogen. The reaction produced the parahydrogenated compounds: (4) (Z)-tert-butyl 4-((2-oxopropanoyl)oxy)but-2-enoate, (5) (Z)-tert-butyl 4-((2-oxopropanoyl)oxy)but-2-enoate-D1, and (6) (Z)-tert-butyl 4-((2-oxopropanoyl)oxy)but-2-enoate-D2, respectively. The singlet relaxation time Tₛ of the resulting compounds (4), (5), and (6) was measured. FIG. 5 shows exemplary singlet lifetimes for the compounds (4), (5), and (6). As shown in FIG. 5, complete deuteration of the PHIP transfer moiety (compound (6)) resulted in a more than 8-fold increase in the singlet relaxation time Tₛ when compared with the non-deuterated PHIP transfer moiety (compound (4)).

Without limitation, the increased singlet relaxation time Tₛ is believed to be the most important factor in the performance increase achieved with fully deuterated compounds described herein. Without limitation, the increased singlet relaxation time Tₛ increases the amount of time in which spin order associated with the parahydrogen can be transferred to other nuclei, such as a ¹³C or ¹⁵N nucleus described herein. Without limitation, such increased polarization transfer times allow for more efficient transfer of spin order to such nuclei.

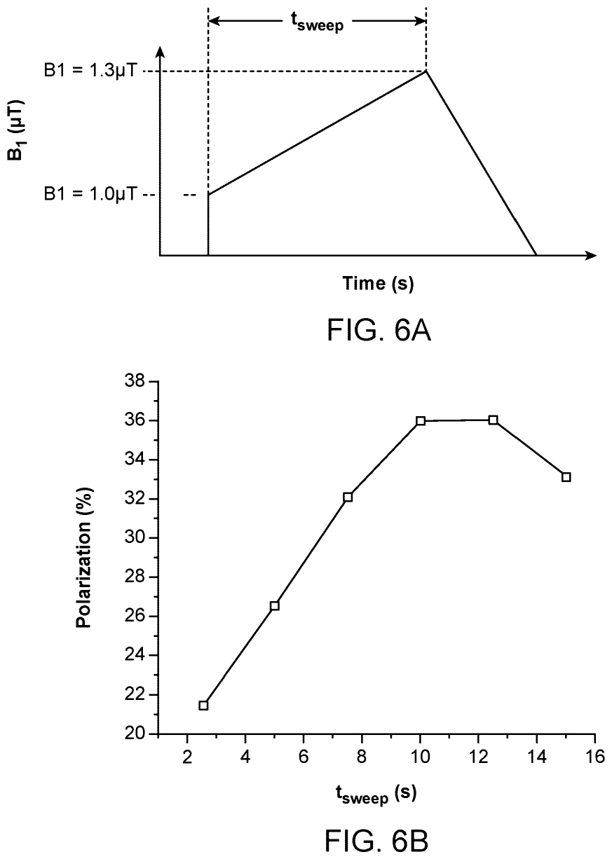

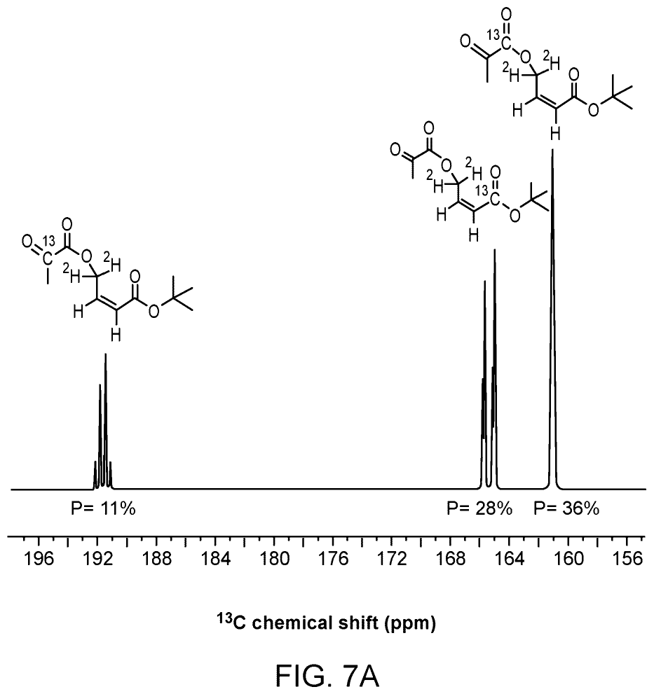

FIG. 6A shows an exemplary polarization transfer pulse sequence used for transferring spin order from the parahydrogen-associated protons in compounds (4), (5), and (6) to the natural abundance ¹³C nuclei present in compounds (4), (5), and (6). Compounds (4), (5), and (6) were placed in a mu-metal magnetic shield and subjected to a magnetic field supplied by a solenoid coil. As shown in FIG. 6A, the strength of the magnetic field B₁ was subjected to a first amplitude modulation by linearly varying the magnetic field strength from 1.0 μT to 1.3 μT. The magnetic field B₁ was linearly polarized and oscillating at the Larmor frequency of the target ¹³C nuclei during the first amplitude modulation. The first amplitude modulation was conducted over a first time period t_{sweep}. During the first amplitude modulation, magnetization in the target ¹³C was built up over the first time period t_{sweep}.

After the first time period t_{sweep}, the strength of the magnetic field Biwas subjected to a second amplitude modulation by linearly varying the magnetic field strength from 1.3 μT to 0 μT. The magnetic field B₁ was linearly detuned away from the Larmor frequency of the target ¹³C nucleus during the second amplitude modulation.

FIG. 6B shows ¹³C polarizations achieved for compound (6) using a variety of first time periods t_{sweep} in the polarization transfer pulse sequence of FIG. 6A. As shown in FIG. 6B, the ¹³C polarization attained a maximum value at a first time period t_{sweep} between about 10 s and about 12 s.

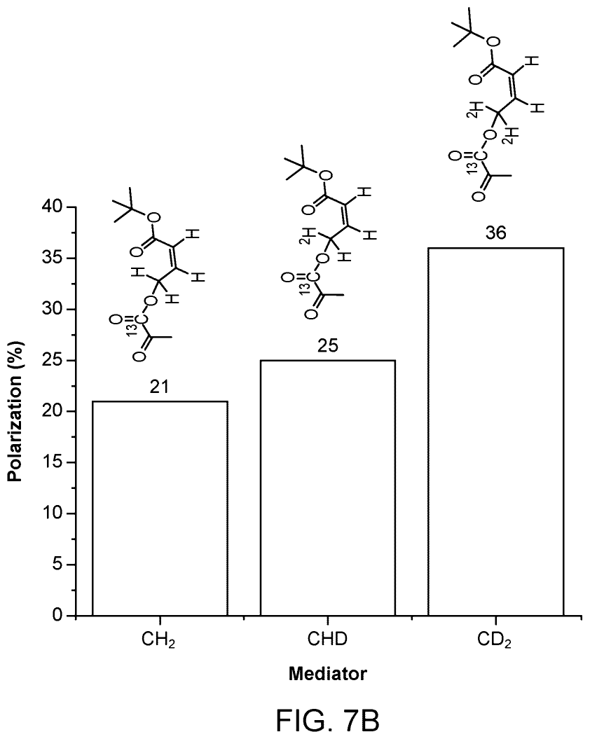

FIG. 7A shows ¹³C polarization levels for compound (6) following the polarization transfer procedure of FIGS. 6A and 6B. As shown in FIG. 7A, ¹³C polarization levels of up to 36% were achieved.

FIG. 7B shows the highest achieved ¹³C polarization levels for compounds (4), (5), and (6). As shown in FIG. 7B, the ¹³C polarization level increased with increasing deuteration.

### Example 2: Polarization Transfer in a Deuterated Lactate Derivative

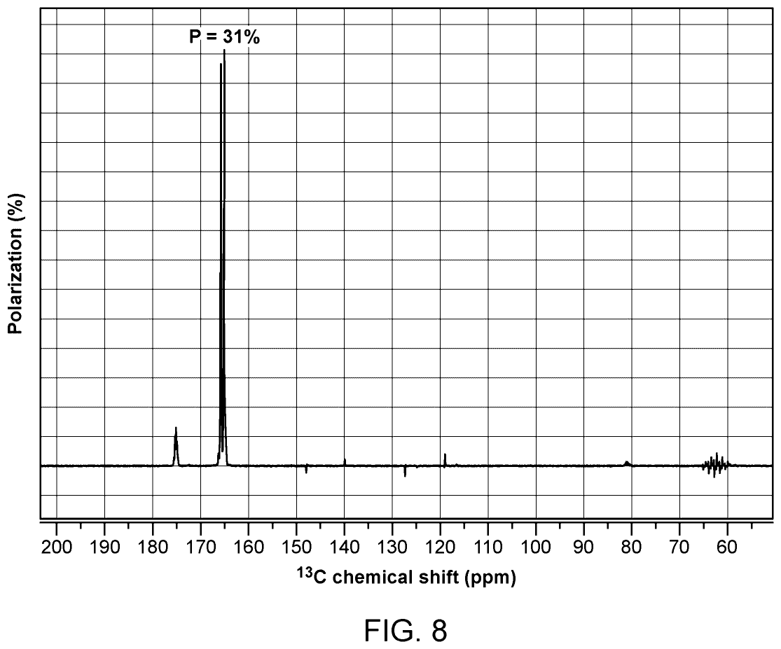

To assess the impact of deuteration level on a PHIP transfer moiety in lactate, compound (7), an ester sidearm derivative of lactate was prepared. Compound (7) was prepared with natural abundance ¹³C (about 1.1%). Hydrogenation of compound (7) was carried out in an NMR pressure tube at 10 bar pressure using hydrogen gas with a >90% parahydrogen enrichment at a flow rate of 0.5 standard liters per minute. 50 mM compound (7) was reacted with the parahydrogen in the present of 2.5 mM Rh(dppb)(COD)BF₄ catalyst solution in acetone-d₆. The reaction produced a parahydrogenated derivative of compound (7). Polarization transfer was carried out in a static field of 50 μT using a transversal magnetic field at the Larmor frequency of ¹³C. This field was ramped from 1.8 μT to 2.2 μT in 9 seconds. FIG. 8 shows ¹³C polarization levels for parahydrogenated compound (7). As shown in FIG. 8, ¹³C polarization levels of up to 310% were achieved.

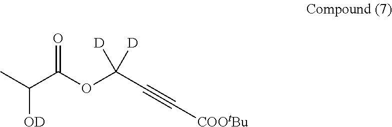

### Example 3: Polarization Transfer in a Deuterated Mono Ethyl Ketoglutarate Derivative

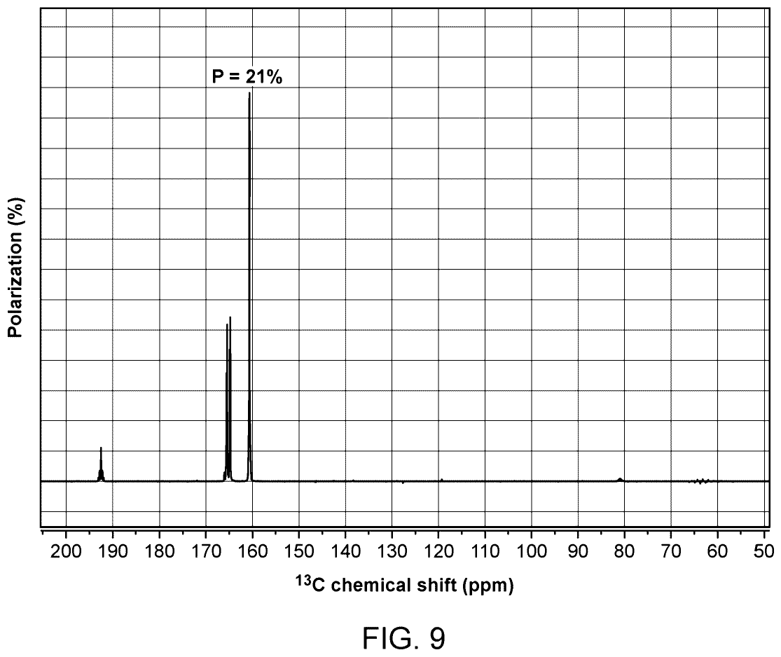

To assess the impact of deuteration level on a PHIP transfer moiety in mono ethyl ketoglutarate, compound (8), an ester sidearm derivative of mono ethyl ketoglutarate was prepared. Compound (8) was prepared with natural abundance ¹³C (about 1.1%). Hydrogenation of compound (8) was carried out in an NMR pressure tube at 10 bar pressure using hydrogen gas with a >90% parahydrogen enrichment at a flow rate of 0.5 standard liters per minute. 50 mM compound (8) was reacted with the parahydrogen in the present of 2.5 mM Rh(dppb)(COD)BF₄ catalyst solution in acetone-d₆. The reaction produced a parahydrogenated derivative of compound (8). Polarization transfer was carried out in a static field of 50 μT using a transversal magnetic field at the Larmor frequency of ¹³C. This field was ramped from 1.8 μT to 2.2 μT in 9 seconds. FIG. 8 shows ¹³C polarization levels for parahydrogenated compound (8). As shown in FIG. 8, ¹³C polarization levels of up to 31% were achieved. As shown in FIG. 9, ¹³C polarization levels of up to 21% were achieved.

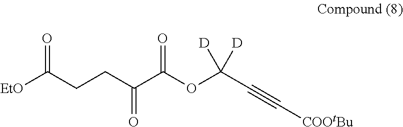

### Example 4: Polarization Transfer in a Z-OMPD Mono Methyl Ester Derivative

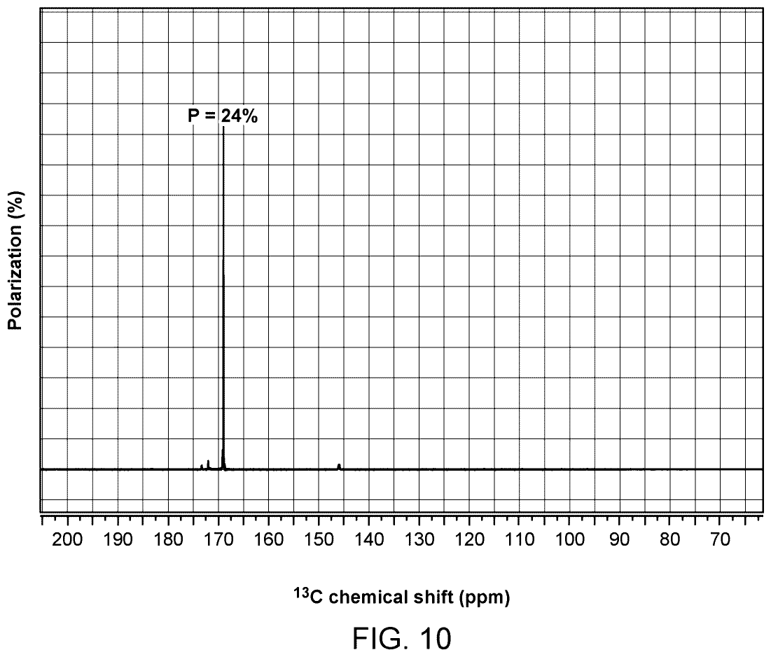

To assess the impact of deuteration level on a PHIP transfer moiety in Z-OMPD mono methyl ester, compound (9), an ester sidearm derivative of Z-OMPD mono methyl ester was prepared. Compound (9) was prepared with natural abundance ¹³C (about 1.1%). Hydrogenation of compound (9) was carried out in an NMR pressure tube at 10 bar pressure using hydrogen gas with a >90% parahydrogen enrichment at a flow rate of 0.5 standard liters per minute. 220 mM compound (9) was reacted with the parahydrogen in the present of 2.5 mM Rh(dppb)(COD)BF₄ catalyst solution in acetone-d₆. The reaction produced a parahydrogenated derivative of compound (9). Polarization transfer was carried out in a static field of 50 μT using a transversal magnetic field at the Larmor frequency of ¹³C. This field was ramped from 1.8 μT to 2.2 μT in 9 seconds. FIG. 8 shows ¹³C polarization levels for parahydrogenated compound (9). FIG. 10 shows ¹³C polarization levels for parahydrogenated compound (9). As shown in FIG. 10, ¹³C polarization levels of up to 24% were achieved.

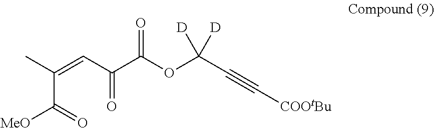

### Recitation of Embodiments

Embodiment 1. A composition comprising a compound of Formula Ia:

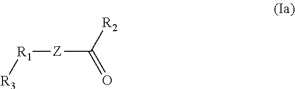

wherein:
Z comprises a carbon-carbon double bond which is fully substituted to include deuterium (D) (—CD=CD-) or a carbon-carbon triple bond (—C≡C—);
R₁ comprises a parahydrogen induced polarization (PHIP) transfer moiety;
R₂ comprises an optionally substituted hydrocarbon or alkoxy group; and
R₃ comprises a biorelevant imaging agent comprising a non-hydrogen nuclear spin.

Embodiment 2. A composition comprising a compound of Formula IIa:

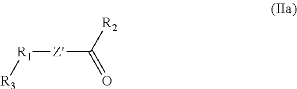

wherein:
Z′ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁ comprises a parahydrogen induced polarization (PHIP) transfer moiety;
R₂ comprises an optionally substituted hydrocarbon or alkoxy group; and
R₃ comprises a biorelevant imaging agent comprising a non-hydrogen nuclear spin.

Embodiment 3. A composition comprising: (i) a biorelevant imaging agent comprising a non-hydrogen nuclear spin; and (ii) a compound of Formula IIIa:

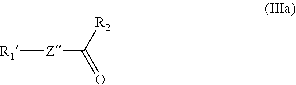

wherein:
Z″ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁′ comprises a parahydrogen induced polarization (PHIP) transfer moiety; and
R₂ comprises an optionally substituted hydrocarbon or alkoxy group.

Embodiment 4. A composition comprising: (i) a hyperpolarized biorelevant imaging agent comprising a non-hydrogen nuclear spin; and (ii) a compound of Formula IVa:

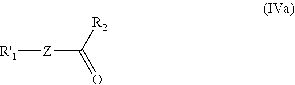

wherein:
Z comprises a carbon-carbon double bond which is fully substituted to include deuterium (—CD=CD-) or a carbon-carbon triple bond (—C≡C—);
R₁′ comprises a parahydrogen induced polarization (PHIP) transfer moiety; and
R₂ comprises an optionally substituted hydrocarbon or alkoxy group.

Embodiment 5. The composition of any one of Embodiments 1-4, wherein the PHIP transfer moiety comprises a fully deuterated C1 hydrocarbon (—CD₂-) or a fully deuterated C2 hydrocarbon (—CD₂-CD₂-).

Embodiment 6. The composition of any one of Embodiments 1-5, wherein:
the PHIP transfer moiety comprises *CR₄R₅, *CR₄Y, *C═Y, or any fully deuterated version thereof;
*C is a ¹²C or ¹³C carbon isotope;
R₄ and R₅ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group; and
Y is selected from the group consisting of a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 7. The composition of any one of Embodiments 1-5, wherein:
the PHIP transfer moiety comprises *CR₆R₇—*CR₈R₉, or any fully deuterated version thereof;
*C is a ¹²C or ¹³C carbon isotope; and
R₆, R₇, R₈, and R₉ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

Embodiment 8. The composition of any one of Embodiments 1-5, wherein:
the PHIP transfer moiety comprises *CH₂, *CH₂—*CH₂, *CHY, *C═Y, or any fully deuterated version thereof;
H* is a hydrogen having a spin order derived from parahydrogen;
*C is a ¹²C or ¹³C carbon isotope; and
Y is selected from the group consisting of a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 9. The composition of Embodiment 6 or 8, wherein the spin-½ atom is chosen from: ¹H, ¹³C ¹⁵N, ¹⁹F, or ³¹P.

Embodiment 10. The composition of any one of Embodiments 1-9, wherein the PHIP transfer moiety includes at least one atom having a J-coupling with the non-hydrogen nuclear spin of at least 0.1 Hertz (Hz).

Embodiment 11. The composition of any one of Embodiments 1-10, wherein Z includes at least one atom having a J-coupling with the non-hydrogen nuclear spin of at least 0.1 Hertz (Hz).

Embodiment 12. The composition of any one of Embodiments 1-11, wherein R₂ comprises a solubilizing moiety.

Embodiment 13. The composition of any one of Embodiments 1-12, wherein R₂ comprises a hydrophobic and/or organophilic moiety.

Embodiment 14. The composition of Embodiment 13, wherein R₂ comprises an organic solubilizing moiety.

Embodiment 15. The composition of any one of Embodiments 1-12, wherein R₂ comprises a hydrophilic and/or organophobic moiety.

Embodiment 16. The composition of any one of Embodiments 1-15, wherein R₂ is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group, a propionic alcohol group, an n-butyl alcohol group, an s-butyl alcohol group, a t-butyl alcohol group, an isobutyl alcohol group, a methoxy group, an ethoxy group, a propoxy group, an isopropoxy group, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, and a tertiary amide group.

Embodiment 17. The composition of any one of Embodiments 1-16, wherein the biorelevant imaging agent comprises a compound of the formula R₁₀C(═O)X—; wherein R₁₀ is chosen from a linear, branched, or cyclic C1-C10 alkyl group, in which one or more C atoms are optionally replaced with C═C, CO, COH, CNH₂, COOH, CH₂COOH, CONH₂, OC(═O); and X is chosen from NR₁₁, S and O; wherein Ru is selected from hydrogen and an amino protecting group, optionally selected from trifluoroacetyl, acetyl, benzoyl, carbobenzoxy, tert-butyl carbonate and benzyl.

Embodiment 18. The composition of any one of Embodiments 1-17, wherein the biorelevant imaging agent is selected from: pyruvate, glutamate, glutamine, lactate, acetate, acetoacetate, zymonate, alanine, fructose, fumarate, bicarbonate, urea, dehydroascorbate, alpha-ketoglutarate, dihydroxyacetone, glucose, ascorbate, and conjugate acids thereof.

Embodiment 19. The composition of any one of Embodiments 1-18, wherein the composition has a solubility in water of less than 50 millimolar (mM).

Embodiment 20. The composition of any one of Embodiments 1-19, wherein reacting the composition with parahydrogen results in a chemical yield of parahydrogenated product of at least 30%.

Embodiment 21. The composition of any one of Embodiments 1-20, for use in a parahydrogen induced polarization (PHIP) process.

Embodiment 22. A method for preparing a hyperpolarized biorelevant imaging agent or a pharmaceutically acceptable salt thereof, the method comprising:
(a) providing a composition comprising a compound of Formula Ia:

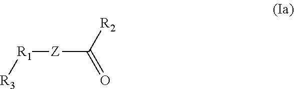

wherein:
   Z comprises a carbon-carbon double bond which is fully substituted to include deuterium (—CD=CD-) or a carbon-carbon triple bond (—C≡C—);
   R₁ comprises a parahydrogen induced polarization (PHIP) transfer moiety;
   R₂ comprises an optionally substituted hydrocarbon or alkoxy group; and
   R₃ comprises a biorelevant imaging agent comprising a non-hydrogen nuclear spin;
   (b) hydrogenating the double bond or the triple bond in the compound of Formula Ia with parahydrogen to form a parahydrogenated derivative of the compound of Formula Ia, the parahydrogenated derivative having the structure of Formula IIa:

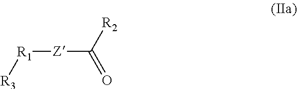

wherein:
   Z′ is a parahydrogenated carbon-carbon single bond which is fully substituted to include deuterium (—CDH*—CDH*—) or a parahydrogenated carbon-carbon double bond (—CH*═CH*—);
   H* is a hydrogen having a spin order derived from parahydrogen;
   R₁ comprises a parahydrogen induced polarization (PHIP) transfer moiety;
   R₂ comprises an optionally substituted hydrocarbon or alkoxy group; and
   R₃ comprises a biorelevant imaging agent comprising a non-hydrogen nuclear spin; and
(c) applying a polarization transferring waveform to transfer nuclear spin order from at least one H* in the compound of Formula IIa to the non-hydrogen nuclear spin, thereby forming a derivative of Formula IIa having a hyperpolarized biorelevant imaging agent.

Embodiment 23. A method for preparing a hyperpolarized biorelevant imaging agent or a pharmaceutically acceptable salt thereof, the method comprising:
(a) providing a composition comprising a compound of Formula IIa:

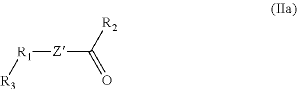

wherein:
   Z′ is a parahydrogenated carbon-carbon single bond which is fully substituted to include deuterium (—CDH*—CDH*—) or a parahydrogenated carbon-carbon double bond (—CH*═CH*—);
   H* is a hydrogen having a spin order derived from parahydrogen;
   R₁ comprises a parahydrogen induced polarization (PHIP) transfer moiety;
   R₂ comprises an optionally substituted hydrocarbon or alkoxy group; and
   R₃ comprises a biorelevant imaging agent comprising a non-hydrogen nuclear spin; and
(b) applying a polarization transferring waveform to transfer nuclear spin order from at least one H* in the compound of Formula IIa to the non-hydrogen nuclear spin, thereby forming a derivative of Formula IIa having a hyperpolarized biorelevant imaging agent.

Embodiment 24. The method of Embodiment 22 or 23, further comprising hydrolyzing the derivative of Formula IIa to provide a composition comprising: (i) a hyperpolarized biorelevant imaging agent comprising a non-hydrogen nuclear spin; and (ii) a compound of Formula IIIa:

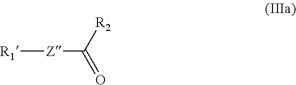

wherein:
Z″ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁′ comprises a parahydrogen induced polarization (PHIP) transfer moiety; and
R₂ comprises an optionally substituted hydrocarbon or alkoxy group.

Embodiment 25. The method of Embodiment 24, further comprising washing the hyperpolarized biorelevant imaging agent one or more times with an organic solvent.

Embodiment 26. The method of Embodiment 25, where the non-hydrogen nuclear spin has a non-hydrogen nuclear spin polarization above 10% after the washing step.

Embodiment 27. The method of any one of Embodiments 22-26, wherein the PHIP transfer moiety comprises a fully deuterated C1 hydrocarbon (—CD₂-) or a fully deuterated C2 hydrocarbon (—CD₂-CD₂-).

Embodiment 28. The method of any one of Embodiments 22-26, wherein:
the PHIP transfer moiety comprises *CR₄R₅, *CR₄Y, *C═Y, or any fully deuterated version thereof,
*C is a ¹²C or ¹³C carbon isotope;
R₄ and R₅ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group; and
Y is selected from: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 29. The method of any one of Embodiments 22-26, wherein:
the PHIP transfer moiety comprises *CR₆R₇—*CR₈R₉, or any fully deuterated version thereof,
*C is a ¹²C or ¹³C carbon isotope; and
R₆, R₇, R₈, and R₉ are each independently selected from the group consisting of: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

Embodiment 30. The method of any one of Embodiments 22-26, wherein:
the PHIP transfer moiety comprises *CH₂, *CH₂—*CH₂, *CHY, *C═Y, or any fully deuterated version thereof,
*C is a ¹²C or ¹³C carbon isotope; and
Y is selected from: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 31. The method of Embodiment 28 or 30, wherein the spin-½ atom is chosen from: ¹H, ¹³C ¹⁵N, ¹⁹F, or ³¹P.

Embodiment 32. The method of any one of Embodiments 22-31, wherein the PHIP transfer moiety includes at least one atom having a J-coupling with the non-hydrogen nuclear spin of at least 0.1 Hertz (Hz).

Embodiment 33. The method of any one of Embodiments 22-32, wherein Z or Z′ includes at least one atom having a J-coupling with the non-hydrogen nuclear spin of at least 0.1 Hertz (Hz).

Embodiment 34. The method of any one of Embodiments 22-33, wherein R₂ comprises a solubilizing moiety.

Embodiment 35. The method of any one of Embodiments 22-34, wherein R₂ comprises a hydrophobic and/or organophilic moiety.

Embodiment 36. The method of Embodiment 35, wherein R₂ comprises an organic solubilizing moiety.

Embodiment 37. The method of any one of Embodiments 22-33, wherein R₂ comprises a hydrophilic and/or organophobic moiety.

Embodiment 38. The method of any one of Embodiments 22-37, wherein R₂ is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group, a propionic alcohol group, an n-butyl alcohol group, an s-butyl alcohol group, a t-butyl alcohol group, an isobutyl alcohol group, a methoxy group, an ethoxy group, a propoxy group, an isopropoxy group, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, and a tertiary amide group.

Embodiment 39. The method of any one of Embodiments 22-38, wherein the biorelevant imaging agent comprises a compound of the formula R₁₀C(═O)X—; wherein R₁₀ is chosen from a linear, branched, or cyclic C1-C10 alkyl group, in which one or more C atoms are optionally replaced with C═C, CO, COH, CNH₂, COOH, CH₂COOH, CONH₂, OC(═O); and X is chosen from NR₁₁, S and O; wherein R₁ is selected from hydrogen and an amino protecting group, optionally selected from trifluoroacetyl, acetyl, benzoyl, carbobenzoxy, tert-butyl carbonate and benzyl.

Embodiment 40. The method of any one of Embodiments 22-39, wherein the biorelevant imaging agent is selected from pyruvate, glutamate, glutamine, lactate, acetate, acetoacetate, zymonate, alanine, fructose, fumarate, bicarbonate, urea, dehydroascorbate, alpha-ketoglutarate, dihydroxyacetone, glucose, ascorbate, and conjugate acids thereof.

Embodiment 41. A hyperpolarized biorelevant imaging agent or a pharmaceutically acceptable salt thereof, produced by the method of any one of Embodiments 22-40.

Embodiment 42. A composition comprising a compound of Formula Ib:

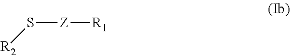

wherein:
Z comprises an ethynyl (—C≡C—) group, a fully deuterated prop-2-ynyl (—CD₂-C≡CD₂-) group, a fully deuterated but-3-ynyl (—CD₂-CD₂-C≡C—) group, a fully deuterated ethenyl (—CD=CD-) group, a fully deuterated prop-2-enyl (—CD₂-CD=CD-) group, or a fully deuterated but-3-enyl (—CD₂-CD₂D₂-CD=CD-) group;
R₁ comprises an optionally substituted hydrocarbon group, alkyl group, cyclic alkyl group, aryl group, carboxyl group, keto group, or alkoxy group; and
R₂ comprises an acyl derivative of a biorelevant imaging agent, the biorelevant imaging agent comprising a non-hydrogen nuclear spin.

Embodiment 43. A composition comprising a compound of Formula IIb:

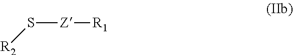

wherein:
Z′ comprises a parahydrogenated ethenyl (—CH*═CH*—) group, a deuterated parahydrogenated prop-2-enyl (—CD₂-CH*═CH*—) group, a deuterated parahydrogenated but-3-enyl (—CD₂-CD₂-CH*═CH*—) group, a deuterated parahydrogenated ethanyl (—CDH*—CDH*—) group, a deuterated parahydrogenated propanyl (—CD₂-CDH*—CDH*—) group, or a deuterated parahydrogenated butanyl (—CD₂-CD₂-CDH*—CDH*—) group;
H* is a hydrogen having a spin order derived from parahydrogen;
R₁ comprises an optionally substituted hydrocarbon group, alkyl group, cyclic alkyl group, aryl group, carboxyl group, keto group, or alkoxy group; and
R₂ comprises an acyl derivative of a biorelevant imaging agent, the biorelevant imaging agent comprising a non-hydrogen nuclear spin.

Embodiment 44. A composition comprising: (i) a biorelevant imaging agent comprising a non-hydrogen nuclear spin; and (ii) a compound of Formula IIIb:

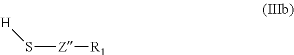

wherein:
Z″ comprises a parahydrogenated ethenyl (—CH*═CH*—) group, a deuterated parahydrogenated prop-2-enyl (—CD₂-CH*═CH*—) group, a deuterated parahydrogenated but-3-enyl (—CD₂-CD₂-CH*═CH*—) group, a deuterated parahydrogenated ethanyl (—CDH*—CDH*—) group, a deuterated parahydrogenated propanyl (—CD₂-CDH*—CDH*—) group, or a deuterated parahydrogenated butanyl (—CD₂-CD₂-CDH*—CDH*—) group;
H* is a hydrogen having a spin order derived from parahydrogen; and
R₁ comprises an optionally substituted hydrocarbon group, alkyl group, cyclic alkyl group, aryl group, carboxyl group, keto group, or alkoxy group.

Embodiment 45. The composition of any one of Embodiments 42-44, wherein the composition further comprises a PHIP transfer moiety between the Z, Z′, and Z″ moiety and the sulfur atom, the PHIP transfer moiety comprising a fully deuterated C1 hydrocarbon (—CD₂-) or a fully deuterated C2 hydrocarbon (—CD₂-CD₂-).

Embodiment 46. The composition of Embodiment 45, wherein:
the PHIP transfer moiety comprises *CR₃R₄, *CR₃Y, *C═Y, or any fully deuterated version thereof;
*C is a ¹²C or ¹³C carbon isotope;
R₃ and R₄ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group; and
Y is selected from the group consisting of: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 47. The composition of Embodiment 45, wherein:
the PHIP transfer moiety comprises*CR₅R₆—*CR₇R₈, or any fully deuterated version thereof,
*C is a ¹²C or ¹³C carbon isotope; and
R₅, R₆, R₇, and R₈ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

Embodiment 48. The composition of Embodiment 45, wherein:
the PHIP transfer moiety comprises *CH₂, *CH₂—*CH₂, *CHY, *C═Y, or any fully deuterated version thereof,
*C is a ¹²C or ¹³C carbon isotope; and
Y is selected from the group consisting of: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 49. The composition of Embodiment 46 or 48, wherein the spin-½ atom is chosen from: ¹H, ¹³C, ¹⁵N, ¹⁹F, or ³¹P.

Embodiment 50. The composition of any one of Embodiments 45-49, wherein the PHIP transfer moiety includes at least one atom having a J-coupling with the non-hydrogen nuclear spin of at least 0.1 Hertz (Hz).

Embodiment 51. The composition of any one of Embodiments 42-50, wherein Z includes at least one atom having a J-coupling with the non-hydrogen nuclear spin of at least 0.1 Hertz (Hz).

Embodiment 52. The composition of any one of Embodiments 42-51, wherein R₁ comprises a solubilizing moiety.

Embodiment 53. The composition of any one of Embodiments 42-52, wherein R₁ comprises a hydrophobic and/or organophilic moiety.

Embodiment 54. The composition of Embodiment 53, wherein R₁ comprises an organic solubilizing moiety.

Embodiment 55. The composition of any one of Embodiments 42-52, wherein R₁ comprises a hydrophilic and/or organophobic moiety.

Embodiment 56. The composition of any one of Embodiments 42-55, wherein R₁ is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group, a propionic alcohol group, an n-butyl alcohol group, an s-butyl alcohol group, a t-butyl alcohol group, an isobutyl alcohol group, a methoxy group, an ethoxy group, a propoxy group, an isopropoxy group, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, a tertiary amide group, and a keto group.

Embodiment 57. The composition of any one of Embodiments 42-56, wherein the biorelevant imaging agent comprises a compound of the formula R₉C(═O)O—; wherein R₉ is chosen from a linear, branched, or cyclic C1-C10 alkyl group, in which one or more C atoms are optionally replaced with C═C, CO, COH, CNH₂, COOH, CH₂COOH, CONH₂, or OC(═O).

Embodiment 58. The composition of any one of Embodiments 42-57, wherein the biorelevant imaging agent is selected from: pyruvate, glutamate, glutamine, lactate, acetate, acetoacetate, zymonate, alanine, fructose, fumarate, bicarbonate, and conjugate acids thereof.

Embodiment 59. The composition of any one of Embodiments 42-58, wherein the composition has a solubility in water of less than 50 millimolar (mM).

Embodiment 60. The composition of any one of Embodiments 42-59, wherein reacting the composition with parahydrogen results in a chemical yield of parahydrogenated product of at least 30%.

Embodiment 61. The composition of any one of Embodiments 42-60, for use in a parahydrogen induced polarization (PHIP) process.

Embodiment 62. A method for preparing a hyperpolarized biorelevant imaging agent or a pharmaceutically acceptable salt thereof, the method comprising:
(a) providing a composition comprising a compound of Formula Ib:

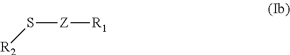

wherein:
   Z comprises an ethynyl (—C≡C—) group, a fully deuterated prop-2-ynyl (—CD₂-C≡C—) group, a fully deuterated but-3-ynyl (—CD₂-CD₂-C≡C—) group, a fully deuterated ethenyl (—CD=CD-) group, a fully deuterated prop-2-enyl (—CD₂-CD=CD-) group, or a fully deuterated but-3-enyl (—CD₂-CD₂-CD=CD-) group;
   R₁ comprises an optionally substituted hydrocarbon group, alkyl group, cyclic alkyl group, aryl group, carboxyl group, keto group, or alkoxy group; and
   R₂ comprises an acyl derivative of a biorelevant imaging agent, the biorelevant imaging agent comprising a non-hydrogen nuclear spin;
(b) hydrogenating the double bond or the triple bond in the compound of Formula Ib with parahydrogen to form a parahydrogenated derivative of the compound of Formula Ib, the parahydrogenated derivative having the structure of Formula IIb:

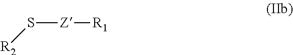

wherein:
   Z′ comprises a parahydrogenated ethenyl (—CH*═CH*—) group, a deuterated parahydrogenated prop-2-enyl (—CD₂-CH*═CH*—) group, a deuterated parahydrogenated but-3-enyl (—CD₂-CD₂-CH*═CH*—) group, a deuterated parahydrogenated ethanyl (—CDH*—CDH*—) group, a deuterated parahydrogenated propanyl (—CD₂-CDH*—CDH*—) group, or a deuterated parahydrogenated butanyl (—CD₂-CD₂-CDH*—CDH*—) group;
   H* is a hydrogen having a spin order derived from parahydrogen;
   R₁ comprises an optionally substituted hydrocarbon group, alkyl group, cyclic alkyl group, aryl group, carboxyl group, keto group, or alkoxy group; and
   R₂ comprises an acyl derivative of a biorelevant imaging agent, the biorelevant imaging agent comprising a non-hydrogen nuclear spin; and
(c) applying a polarization transferring waveform to transfer nuclear spin order from at least one H* in the compound of Formula IIb to the non-hydrogen nuclear spin, thereby forming a derivative of Formula IIb having a hyperpolarized acyl derivative of the biorelevant imaging agent.

Embodiment 63. A method for preparing a hyperpolarized biorelevant imaging agent or a pharmaceutically acceptable salt thereof, the method comprising:
(a) providing a composition comprising a compound of Formula IIb:

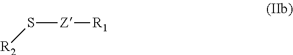

wherein:
   Z′ comprises a parahydrogenated ethenyl (—CH*═CH*—) group, a deuterated parahydrogenated prop-2-enyl (—CD₂-CH*═CH*—) group, a deuterated parahydrogenated but-3-enyl (—CD₂-CD₂-CH*═CH*—) group, a deuterated parahydrogenated ethanyl (—CDH*—CDH*—) group, a deuterated parahydrogenated propanyl (—CD₂-CDH*—CDH*—) group, or a deuterated parahydrogenated butanyl (—CD₂-CD₂-CDH*—CDH*—) group;
   H* is a hydrogen having a spin order derived from parahydrogen;
   R₁ comprises an optionally substituted hydrocarbon group, alkyl group, cyclic alkyl group, aryl group, carboxyl group, keto group, or alkoxy group; and
   R₂ comprises an acyl derivative of a biorelevant imaging agent, the biorelevant imaging agent comprising a non-hydrogen nuclear spin; and
(b) applying a polarization transferring waveform to transfer nuclear spin order from at least one H* in the compound of Formula IIb to the non-hydrogen nuclear spin, thereby forming a derivative of Formula IIb having a hyperpolarized acyl derivative of the biorelevant imaging agent.

Embodiment 64. The method of Embodiment 62 or 63, further comprising hydrolyzing the derivative of Formula IIb to provide a composition comprising: (i) a hyperpolarized biorelevant imaging agent comprising a non-hydrogen nuclear spin; and (ii) a compound of Formula IIIb:

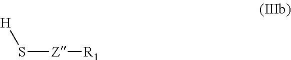

wherein:
Z″ comprises a parahydrogenated ethenyl (—CH*═CH*—) group, a deuterated parahydrogenated prop-2-enyl (—CD₂-CH*═CH*—) group, a deuterated parahydrogenated but-3-enyl (—CD₂-CD₂-CH*═CH*—) group, a deuterated parahydrogenated ethanyl (—CDH*—CDH*—) group, a deuterated parahydrogenated propanyl (—CD₂-CDH*—CDH*—) group, or a deuterated parahydrogenated butanyl (—CD₂-CD₂-CDH*—CDH*—) group;
H* is a hydrogen having a spin order derived from parahydrogen; and
R₁ comprises an optionally substituted hydrocarbon group, alkyl group, cyclic alkyl group, aryl group, carboxyl group, keto group, or alkoxy group.

Embodiment 65. The method of Embodiment 64, further comprising washing the hyperpolarized biorelevant imaging agent one or more times with an organic solvent.

Embodiment 66. The method of Embodiment 65, where the non-hydrogen nuclear spin has a non-hydrogen nuclear spin polarization above 10% after the washing step.

Embodiment 67. The method of any one of Embodiments 62-66, wherein the composition further comprises a PHIP transfer moiety between the Z, Z′, or Z″ moiety and the sulfur atom, the PHIP transfer moiety comprising a fully deuterated C1 hydrocarbon (—CD₂-) or a fully deuterated C2 hydrocarbon (—CD₂-CD₂-).

Embodiment 68. The method of Embodiment 67, wherein:
the PHIP transfer moiety comprises *CR₃R₄, *CR₃Y, *C═Y, or any fully deuterated version thereof;
*C is a ¹²C or ¹³C carbon isotope;
R₃ and R₄ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group; and
Y is selected from: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 69. The method of Embodiment 67, wherein:
the PHIP transfer moiety comprises *CR₅R₆—*CR₇R₈, or any fully deuterated version thereof,
*C is a ¹²C or ¹³C carbon isotope; and
R₅, R₆, R₇, and R₈ are each independently selected from the group consisting of: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

Embodiment 70. The method of Embodiment 67, wherein:
the PHIP transfer moiety comprises *CH₂, *CH₂—*CH₂, *CHY, *C═Y, or any fully deuterated version thereof,
*C is a ¹²C or ¹³C carbon isotope; and
Y is selected from: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

Embodiment 71. The method of Embodiment 68 or 70, wherein the spin-½ atom is chosen from: ¹H, ¹³C, ¹⁵N, ¹⁹F, or ³¹P.

Embodiment 72. The method of any one of Embodiments 67-71, wherein the PHIP transfer moiety includes at least one atom having a J-coupling with the non-hydrogen nuclear spin of at least 0.1 Hertz (Hz).

Embodiment 73. The method of any one of Embodiments 62-72, wherein Z or Z′ includes at least one atom having a J-coupling with the non-hydrogen nuclear spin of at least 0.1 Hertz (Hz).

Embodiment 74. The method of any one of Embodiments 62-73, wherein R₁ comprises a solubilizing moiety.

Embodiment 75. The method of any one of Embodiments 62-74, wherein R₁ comprises a hydrophobic and/or organophilic moiety.

Embodiment 76. The method of Embodiment 75, wherein R₁ comprises an organic solubilizing moiety.

Embodiment 77. The method of any one of Embodiments 62-73, wherein R₁ comprises a hydrophilic and/or organophobic moiety.

Embodiment 78. The method of any one of Embodiments 62-77, wherein R₁ is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group, a propionic alcohol group, an n-butyl alcohol group, an s-butyl alcohol group, a t-butyl alcohol group, an isobutyl alcohol group, a methoxy group, an ethoxy group, a propoxy group, an isopropoxy group, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, a tertiary amide group, and a keto group.

Embodiment 79. The method of any one of Embodiments 62-78, wherein the biorelevant imaging agent comprises a compound of the formula R₉C(═O)O—; wherein R₉ is chosen from a linear, branched, or cyclic C1-C10 alkyl group, in which one or more C atoms are optionally replaced with C═C, CO, COH, CNH₂, COOH, CH₂COOH, CONH₂, OC(═O).

Embodiment 80. The method of any one of Embodiments 62-79, wherein the biorelevant imaging agent is selected from pyruvate, glutamate, glutamine, lactate, acetate, acetoacetate, zymonate, alanine, fructose, fumarate, bicarbonate, and conjugate acids thereof.

Embodiment 81. A hyperpolarized biorelevant imaging agent or a pharmaceutically acceptable salt thereof, produced by the method of any one of Embodiments 62-80.

What is claimed is:

1. A composition comprising a compound of Formula Ia: 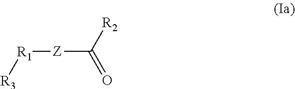
wherein:
Z comprises a carbon-carbon double bond which is fully substituted to include deuterium (D) (—CD=CD-) or a carbon-carbon triple bond (—C≡C—);
R₁ comprises a parahydrogen induced polarization (PHIP) transfer moiety;
R₂ comprises an optionally substituted hydrocarbon or alkoxy group; and
R₃ comprises a biorelevant imaging agent comprising a non-hydrogen nuclear spin.

2. A composition comprising a compound of Formula IIa: 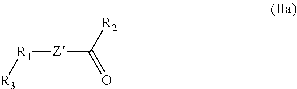
wherein:
Z′ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁ comprises a parahydrogen induced polarization (PHIP) transfer moiety;
R₂ comprises an optionally substituted hydrocarbon or alkoxy group; and
R₃ comprises a biorelevant imaging agent comprising a non-hydrogen nuclear spin.

3. A composition comprising: (i) a biorelevant imaging agent comprising a non-hydrogen nuclear spin; and (ii) a compound of Formula IIIa: 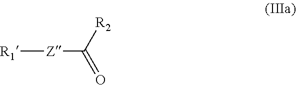
wherein:
Z″ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁′ comprises a parahydrogen induced polarization (PHIP) transfer moiety; and
R₂ comprises an optionally substituted hydrocarbon or alkoxy group.

4. A composition comprising: (i) a hyperpolarized biorelevant imaging agent comprising a non-hydrogen nuclear spin; and (ii) a compound of Formula IVa: 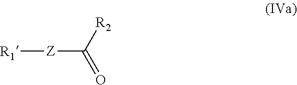
wherein:
Z comprises a carbon-carbon double bond which is fully substituted to include deuterium (—CD=CD-) or a carbon-carbon triple bond (—C≡C—);
R₁′ comprises a parahydrogen induced polarization (PHIP) transfer moiety; and
R₂ comprises an optionally substituted hydrocarbon or alkoxy group.

5. The composition of any one of claims 1-4, wherein the PHIP transfer moiety comprises a fully deuterated C1 hydrocarbon (—CD₂-) or a fully deuterated C2 hydrocarbon (—CD₂-CD₂-).

6. The composition of any one of claims 1-5, wherein:
the PHIP transfer moiety comprises *CR₄R₅, *CR₄Y, *C═Y, or any fully deuterated version thereof;
*C is a ¹²C or ¹³C carbon isotope;
R₄ and R₅ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group; and
Y is selected from the group consisting of: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

7. The composition of any one of claims 1-5, wherein:
the PHIP transfer moiety comprises *CR₆R₇—*CR₈R₉, or any fully deuterated version thereof,
*C is a ¹²C or ¹³C carbon isotope; and
R₆, R₇, R₈, and R₉ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

8. The composition of any one of claims 1-5, wherein:
the PHIP transfer moiety comprises *CH₂, *CH₂—*CH₂, *CHY, *C═Y, or any fully deuterated version thereof,
*C is a ¹²C or ¹³C carbon isotope; and
Y is selected from the group consisting of: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

9. The composition of claim 6 or 8, wherein the spin-½ atom is chosen from: ¹H, ¹³C ¹⁵N, ¹⁹F, or ³¹P.

10. The composition of any one of claims 1-9, wherein the PHIP transfer moiety includes at least one atom having a J-coupling with the non-hydrogen nuclear spin of at least 0.1 Hertz (Hz).

11. The composition of any one of claims 1-10, wherein Z includes at least one atom having a J-coupling with the non-hydrogen nuclear spin of at least 0.1 Hertz (Hz).

12. The composition of any one of claims 1-11, wherein R₂ comprises a solubilizing moiety.

13. The composition of any one of claims 1-12, wherein R₂ comprises a hydrophobic and/or organophilic moiety.

14. The composition of claim 13, wherein R₂ comprises an organic solubilizing moiety.

15. The composition of any one of claims 1-12, wherein R₂ comprises a hydrophilic and/or organophobic moiety.

16. The composition of any one of claims 1-15, wherein R₂ is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group, a propionic alcohol group, an n-butyl alcohol group, an s-butyl alcohol group, a t-butyl alcohol group, an isobutyl alcohol group, a methoxy group, an ethoxy group, a propoxy group, an isopropoxy group, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, and a tertiary amide group.

17. The composition of any one of claims 1-16, wherein the biorelevant imaging agent comprises a compound of the formula R₁₀C(═O)X—; wherein R₁₀ is chosen from a linear, branched, or cyclic C1-C10 alkyl group, in which one or more C atoms are optionally replaced with C═C, CO, COH, CNH₂, COOH, CH₂COOH, CONH₂, OC(═O); and X is chosen from NR₁₁, S and O; wherein R₁₁ is selected from hydrogen and an amino protecting group, optionally selected from trifluoroacetyl, acetyl, benzoyl, carbobenzoxy, tert-butyl carbonate and benzyl.

18. The composition of any one of claims 1-17, wherein the biorelevant imaging agent is selected from: pyruvate, glutamate, glutamine, lactate, acetate, acetoacetate, zymonate, alanine, fructose, fumarate, bicarbonate, urea, dehydroascorbate, alpha-ketoglutarate, dihydroxyacetone, glucose, ascorbate, and conjugate acids thereof.

19. The composition of any one of claims 1-18, wherein the composition has a solubility in water of less than 50 millimolar (mM).

20. The composition of any one of claims 1-19, wherein reacting the composition with parahydrogen results in a chemical yield of parahydrogenated product of at least 30%.

21. The composition of any one of claims 1-20, for use in a parahydrogen induced polarization (PHIP) process.

22. A method for preparing a hyperpolarized biorelevant imaging agent or a pharmaceutically acceptable salt thereof, the method comprising:
(a) providing a composition comprising a compound of Formula Ia: 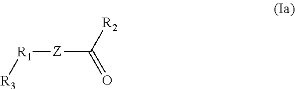
wherein:
Z comprises a carbon-carbon double bond which is fully substituted to include deuterium (—CD=CD-) or a carbon-carbon triple bond (—C≡C—);
R₁ comprises a parahydrogen induced polarization (PHIP) transfer moiety;
R₂ comprises an optionally substituted hydrocarbon or alkoxy group; and
R₃ comprises a biorelevant imaging agent comprising a non-hydrogen nuclear spin;
(b) hydrogenating the double bond or the triple bond in the compound of Formula Ia with parahydrogen to form a parahydrogenated derivative of the compound of Formula Ia, the parahydrogenated derivative having the structure of Formula IIa: 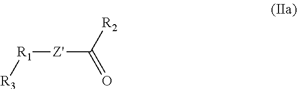
wherein:
Z′ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁ comprises a parahydrogen induced polarization (PHIP) transfer moiety;
R₂ comprises an optionally substituted hydrocarbon or alkoxy group; and
R₃ comprises a biorelevant imaging agent comprising a non-hydrogen nuclear spin; and
(c) applying a polarization transferring waveform to transfer nuclear spin order from at least one H* in the compound of Formula IIa to the non-hydrogen nuclear spin, thereby forming a derivative of Formula IIa having a hyperpolarized biorelevant imaging agent.

23. A method for preparing a hyperpolarized biorelevant imaging agent or a pharmaceutically acceptable salt thereof, the method comprising:
(a) providing a composition comprising a compound of Formula IIa: 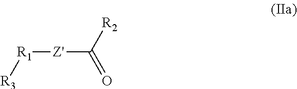
wherein:
Z′ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁ comprises a parahydrogen induced polarization (PHIP) transfer moiety;
R₂ comprises an optionally substituted hydrocarbon or alkoxy group; and
R₃ comprises a biorelevant imaging agent comprising a non-hydrogen nuclear spin; and
(b) applying a polarization transferring waveform to transfer nuclear spin order from at least one H* in the compound of Formula IIa to the non-hydrogen nuclear spin, thereby forming a derivative of Formula IIa having a hyperpolarized biorelevant imaging agent.

24. The method of claim 22 or 23, further comprising hydrolyzing the derivative of Formula IIa to provide a composition comprising: (i) a hyperpolarized biorelevant imaging agent comprising a non-hydrogen nuclear spin; and (ii) a compound of Formula IIIa: 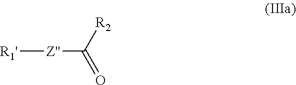
wherein:
Z″ is a parahydrogenated carbon-carbon single bond which is fully substituted to include deuterium (—CDH*—CDH*—) or a parahydrogenated carbon-carbon double bond (—CH*═CH*—);
H* is a hydrogen having a spin order derived from parahydrogen;
R₁′ comprises a parahydrogen induced polarization (PHIP) transfer moiety; and
R₂ comprises an optionally substituted hydrocarbon or alkoxy group.

25. The method of claim 24, further comprising washing the hyperpolarized biorelevant imaging agent one or more times with an organic solvent.

26. The method of claim 25, where the non-hydrogen nuclear spin has a non-hydrogen nuclear spin polarization above 10% after the washing step.

27. The method of any one of claims 22-26, wherein the PHIP transfer moiety comprises a fully deuterated C1 hydrocarbon (—CD₂-) or a fully deuterated C2 hydrocarbon (—CD₂-CD₂-).

28. The method of any one of claims 22-26, wherein:
the PHIP transfer moiety comprises *CR₄R₅, *CR₄Y, *C═Y, or any fully deuterated version thereof;
*C is a ¹²C or ¹³C carbon isotope;
R₄ and R₅ are each independently selected from: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group; and
Y is selected from: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

29. The method of any one of claims 22-26, wherein:
the PHIP transfer moiety comprises *CR₆R₇—*CR₈R₉, or any fully deuterated version thereof,
*C is a ¹²C or ¹³C carbon isotope; and
R₆, R₇, R₈, and R₉ are each independently selected from the group consisting of: deuterium, a fully deuterated linear, branched, or cyclic C1-C10 alkyl hydrocarbon, a fully deuterated C6 aryl, a fully deuterated benzyl, a fully deuterated phenyl, a fully deuterated heteroaryl, and a fully deuterated haloalkyl group.

30. The method of any one of claims 22-26, wherein:
the PHIP transfer moiety comprises *CH₂, *CH₂—*CH₂, *CHY, *C═Y, or any fully deuterated version thereof,
*C is a ¹²C or ¹³C carbon isotope; and
Y is selected from: a spin-½ atom, and a spin-½ atom covalently bonded to one or more chemical moiety chosen from: a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group, or a heteroatom such as N, O, S, optionally substituted with a fully deuterated linear, branched, or cyclic C1-C10 alkyl hydrocarbon, a fully deuterated C6 aryl, fully deuterated benzyl, fully deuterated phenyl, fully deuterated heteroaryl, halogen or fully deuterated haloalkyl group.

31. The method of claim 28 or 30, wherein the spin-½ atom is chosen from: ¹H, ¹³C ¹⁵N, ¹⁹F, or ³¹P.

32. The method of any one of claims 22-31, wherein the PHIP transfer moiety includes at least one atom having a J-coupling with the non-hydrogen nuclear spin of at least 0.1 Hertz (Hz).

33. The method of any one of claims 22-32, wherein Z or Z′ includes at least one atom having a J-coupling with the non-hydrogen nuclear spin of at least 0.1 Hertz (Hz).

34. The method of any one of claims 22-33, wherein R₂ comprises a solubilizing moiety.

35. The method of any one of claims 22-34, wherein R₂ comprises a hydrophobic and/or organophilic moiety.

36. The method of claim 35, wherein R₂ comprises an organic solubilizing moiety.

37. The method of any one of claims 22-33, wherein R₂ comprises a hydrophilic and/or organophobic moiety.

38. The method of any one of claims 22-37, wherein R₂ is selected from: a methyl group, an ethyl group, a propyl group, an isopropyl group, an n-butyl group, an s-butyl group, a t-butyl group, an isobutyl group, a hydroxy group, a methyl alcohol group, an ethyl alcohol group, an n-propanol group, an isopropyl alcohol group, a propionic alcohol group, an n-butyl alcohol group, an s-butyl alcohol group, a t-butyl alcohol group, an isobutyl alcohol group, a methoxy group, an ethoxy group, a propoxy group, an isopropoxy group, a propionic group, a butoxy group, a t-butoxy group, a s-butoxy group, an ester group, a phenyl group, a substituted phenyl group, a primary amine group, a secondary amine group, a tertiary amine group, a primary amide group, a secondary amide group, and a tertiary amide group.

39. The method of any one of claims 22-38, wherein the biorelevant imaging agent comprises a compound of the formula R₁₀C(═O)X—; wherein R₁₀ is chosen from a linear, branched, or cyclic C1-C10 alkyl group, in which one or more C atoms are optionally replaced with C═C, CO, COH, CNH₂, COOH, CH₂COOH, CONH₂, OC(═O); and X is chosen from NR₁₁, S and O; wherein R₁₁ is selected from hydrogen and an amino protecting group, optionally selected from trifluoroacetyl, acetyl, benzoyl, carbobenzoxy, tert-butyl carbonate and benzyl.

40. The method of any one of claims 22-39, wherein the biorelevant imaging agent is selected from pyruvate, glutamate, glutamine, lactate, acetate, acetoacetate, zymonate, alanine, fructose, fumarate, bicarbonate, urea, dehydroascorbate, alpha-ketoglutarate, dihydroxyacetone, glucose, ascorbate, and conjugate acids thereof.

41. A hyperpolarized biorelevant imaging agent or a pharmaceutically acceptable salt thereof, produced by the method of any one of claims 22-40.
